(19) United States
(12) Patent Application Publication
(10) Pub. No.: US 2026/0286284 A1
(43) Pub. Date: Sep. 24, 2026

(54) GLUCOLIPID PRODUCTION

(71) Applicant: Evonik Operations GmbH, Essen (DE)

(72) Inventors: Steffen SCHAFFER, Herten (DE); Mirja WESSEL, Bochum (DE); Hans Henning WENK, Mülheim an der Ruhr (DE); Maik OLFERT, Datteln (DE)

(73) Assignee: Evonik Operations GmbH, Essen (DE)

(21) Appl. No.: 19/482,478

(22) PCT Filed: May 2, 2024
(86) PCT No.: PCT/EP2024/062077

§ 371 Date: Nov. 7, 2025

(30) Foreign Application Priority Data
May 11, 2023 (EP) 23172836.1

(51) Int. Cl. C12N 1/20; C12N 9/10; C12N 15/78; C12P 19/18; C12P 19/44; C12R 1/40

(52) U.S. Cl. CPC C12N 1/20; C12N 9/1048; C12N 9/1051; C12N 15/78; C12P 19/18; C12P 19/44; C12R 2001/40; C12Y 204/01212

(57) ABSTRACT

A microbial cell for producing at least one lipid with the following general formula II from at least one carbon substrate is provided,

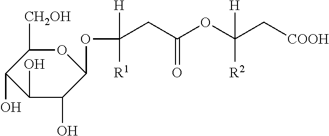

wherein R¹ and R² independently of one another comprises identical or different organic radicals each with 5 to 13 carbon atoms, and the cell is a non-pathogenic cell that is genetically modified to increase the heterologous expression relative to the wild-type cell of: Enzyme E₁ a 3-(3-hydroxyalkanoyloxy)alkanoic acid (HAA) synthase comprising SEQ ID NO: 1, SEQ ID NO: 7, SEQ ID NO:11, SEQ ID NO: 15 or a variant thereof, wherein the variant comprises 60% sequence identity to SEQ ID NO: 1, 7, 11 or 15; and Enzyme E₂ a glycosyltransferase (EC 2.4) comprising SEQ ID NO: 2, SEQ ID NO: 8, or SEQ ID NO: 12, or a variant thereof, wherein the variant comprises 60% sequence identity to SEQ ID NO: 2, 8 or 12.

### FIELD OF THE INVENTION

The present invention relates to a recombinant cell and a biotechnological method for producing glucolipids. In particular, the cell is a non-pathogenic cell genetically modified to produce at least one glucolipid.

### BACKGROUND OF THE INVENTION

Today, most of the available surfactants such as Sodium Laureth Sulfate (SLES), betaine and the like are produced chemically at an industrial scale. These chemically produced surfactants have all the disadvantages that usually come with the use of a chemical production process such as the formation of harmful byproducts. For example, in the SLES production process, at least one harmful byproduct 1,4-dioxane is produced. In order to reduce the amount of toxic products generated and in view of consumers' increasing demand for environmentally friendly products, there is a general trend towards production and use of biosurfactants. Besides producing less poisonous byproducts during the manufacture process, biosurfactants also have useful properties like high structural diversity, beneficial surfactant properties, low environmental toxicity, antibiotic or bioactive properties and complete biological degradability. There is thus a general impetus towards producing and using biosurfactants instead of chemical surfactants.

Rubiwettins, a group of glucolipids, are at least one example of such a biosurfactant. Rubiwettins represent an economically interesting class of surfactants because they may potentially replace chemically produced surfactants.

Rubiwettins are exolipids composed of one β-D-glucose molecule linked to a 3-hydroxy fatty acid dimer with hydroxy fatty acid chain lengths between C₁₄ and C₈ as lipid main components. They have surface-active properties. Rubiwettins are currently being synthesized by a wildtype Serratia rubidaea isolate which is a human- and animal pathogen. The fact that this production organism is able to cause diseases considerably reduces the customer acceptance for these conventionally produced rubiwettins. Further, higher safety requirements are also needed during the production process of rubiwettins and this increases the costs owing to increased capital expenditure and possibly additional production steps.

The current methods available for production of biosurfactants such as rubiwettins involve the use of these pathogenic organisms. The yield of production can be optimized by varying pH, oxygen supply, media composition, feeding strategies, nitrogen supply, temperature, choice of substrate and the like. However, if rubiwettins are to be employed on a large scale as surfactants, they will have to compete with the currently employed surfactants. The latter are bulk chemicals, which can be produced at a very low cost. Therefore, rubiwettins must also be produced at costs as low as possible, without health risks for the customer and with defined properties as far as possible. This is not possible by merely optimizing the performance parameters via process optimization.

WO 2019/154984 discloses a non-pathogenic cell that is genetically modified to introduce specific enzymes that are able to produce rubiwettins from a carbon substrate. However, there is still a need in the art for an alternative more efficient method of producing rubiwettins with even higher product yields.

### DESCRIPTION OF THE INVENTION

The present invention attempts to solve the problems above by providing a biotechnological means of producing biosurfactants such as lipids, in particular rubiwettins from a carbon source using a non-pathogenic cell. In particular, the non-pathogenic cell may be genetically modified to increase the expression of at least one enzyme (E₂) that is capable of converting 3-hydroxyalkanoyl-3-hydroxyalkanoyl-CoA/ACP and/or 3-(3-hydroxyalkanoyloxy)alkanoic acid (HAA) in combination with NDP-glucose into β-D-glucopyranosyl-3-hydroxyalkanoyl-3-hydroxyalkanoate, wherein the enzyme E₂ is a glycosyltransferase (EC 2.4) comprising SEQ ID NO: 1, SEQ ID NO: 7, SEQ ID NO:11 or SEQ ID NO: 15, or a variant thereof. The genetically modified cell may then be used to convert a suitable carbon source to a lipid with general formula II below:

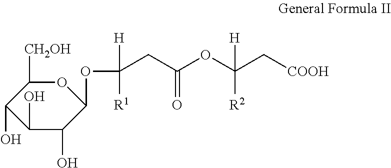

wherein R¹ and R² independently of one another is an identical or different alkyl group with 5 to 13 carbon atoms. In particular, the alkyl group may be saturated or unsaturated. More in particular, the alkyl group of R¹ and/or R² may be a saturated alkyl radical. Even more in particular, R¹ and/or R² may be selected from the group consisting of pentyl, heptyl, nonyl, undecyl, tridecyl and (CH₂)ₙ—CH₃ with n=4-12.

In particular, the cell may be further genetically modified to increase the expression of at least one enzyme (E₁) capable of converting 3-hydroxyalkanoyl-CoA/ACP into 3-hydroxyalkanoyl-3-hydroxyalkanoyl-CoA/ACP and further to 3-(3-hydroxyalkanoyloxy)alkanoic acid (HAA), wherein the enzyme E₁ may be a 3-(3-hydroxyalkanoyloxy)alkanoic acid (HAA) synthase comprising SEQ ID NO: 1, SEQ ID NO: 7, SEQ ID NO; 11, SEQ ID NO: 15, or a variant thereof.

The cell according to any aspect of the present invention may be capable of producing rubiwettins from a carbon source using a non-pathogenic cell.

This lipid of General Formula II may also be known as a glucolipid and more particularly a rubiwettin or a glucolipid. The genetically modified cell according to any aspect of the present invention has the advantage of being non-pathogenic and simple to culture. This enables the cell to be safer for production and also keeps the costs lower as no special safety requirements are needed in the lab during production and use of the rubiwettins. The cells according to any aspect of the present invention has the further advantage of being able to use a variety of carbon substrates to produce the lipids according to any aspect of the present invention. For example simple carbons such as glucose may be used as a carbon substrate. Also, the lipids formed according to any aspect of the present invention have defined and flexible properties. A further advantage is that rubiwettins can be produced with higher space-time yield, higher carbon yields, product concentration, product homogeneity (fatty acid species) than with cells without enhancement of these activities.

According to one aspect of the present invention, there is provided a microbial cell for producing at least one lipid with general formula II from at least one carbon substrate,

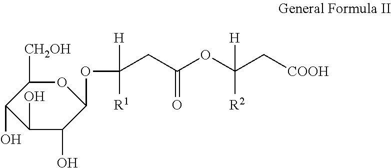

wherein R¹ and R² independently of one another comprises identical or different organic radicals each with 5 to 13 carbon atoms,
wherein the cell is a non-pathogenic cell that is genetically modified to increase the heterologous expression relative to the wild-type cell of:
   Enzyme E₁ a 3-(3-hydroxyalkanoyloxy)alkanoic acid (HAA) synthase comprising SEQ ID NO: 1, SEQ ID NO: 7, SEQ ID NO: 11, SEQ ID NO: 15 or a variant thereof, wherein the variant comprises 60% sequence identity to SEQ ID NO: 1, 7, 11 or 15; and
   Enzyme E₂ a glycosyltransferase (EC 2.4) comprising SEQ ID NO: 2, SEQ ID NO: 8, or SEQ ID NO: 12, or a variant thereof, wherein the variant comprises 60% sequence identity to SEQ ID NO:2, 8 or 12.

In particular, glucose may be added for either or both of the conversions to take place. Glucose may thus be added for the conversion of 3-hydroxyalkanoyl-3-hydroxyalkanoyl-CoA/ACP into β-D-glucopyranosyl-3-hydroxyalkanoyl-3-hydroxyalkanoate. In another example, glucose may be added for conversion of HAA into β-D-glucopyranosyl-3-hydroxyalkanoyl-3-hydroxyalkanoate. In another example, both HAA and 3-hydroxyalkanoyl-3-hydroxyalkanoyl-CoA/ACP may be present at the same time for β-D-glucopyranosyl-3-hydroxyalkanoyl-3-hydroxyalkanoate production.

The cell according to any aspect of the present invention may be genetically modified to increase the heterologous expression relative to the wild type cell of enzyme (E₁) which is capable of converting 3-hydroxyalkanoyl-CoA/ACP into 3-hydroxyalkanoyl-3-hydroxyalkanoyl-CoA/ACP and further to 3-(3-hydroxyalkanoyloxy)alkanoic acid (HAA). In particular, the enzyme E₁ may be a 3-(3-hydroxyalkanoyloxy)alkanoic acid (HAA) synthase. In one example, the enzyme E₁ comprises a sequence selected from the group consisting of SEQ ID NO: 1, 7, 11, 15 or variant thereof. In one example, the variant of SEQ ID NO: 1, 7, 11, or 15 comprises 60% sequence identity to SEQ ID NO: 1, 7, 11 or 15 respectively.

In one example, the enzyme E₁ may have polypeptide sequence SEQ ID NO: 1 or a polypeptide sequence in which up to 25%, preferably up to 20%, particularly preferably up to 15% in particular up to 10, 9, 8, 7, 6, 5, 4, 3, 2, 1% of the amino acid radicals are modified compared to the reference sequence SEQ ID NO: 1 by deletion, insertion, substitution or a combination thereof and that still has at least 10%, particularly 50%, more particularly 80%, even more particularly more than 90% of the enzymatic activity of the enzyme having the reference sequence SEQ ID NO: 1, wherein enzymatic activity for an enzyme E₁ is understood as meaning the ability preferably to convert 3-hydroxyalkanoyl-CoA/ACP into 3-hydroxyalkanoyl-3-hydroxyalkanoyl-CoA/ACP and further to HAA.

In another example, the enzyme E₁ may have polypeptide sequence SEQ ID NO: 7 or a polypeptide sequence in which up to 25%, preferably up to 20%, particularly preferably up to 15% in particular up to 10, 9, 8, 7, 6, 5, 4, 3, 2, 1% of the amino acid radicals are modified compared to the reference sequence SEQ ID NO: 7 by deletion, insertion, substitution or a combination thereof and that still has at least 10%, particularly 50%, more particularly 80%, even more particularly more than 90% of the enzymatic activity of the enzyme having the reference sequence SEQ ID NO: 7, wherein enzymatic activity for an enzyme E₁ is understood as meaning the ability preferably to convert 3-hydroxyalkanoyl-CoA/ACP into 3-hydroxyalkanoyl-3-hydroxyalkanoyl-CoA/ACP and further to HAA.

In a further example, the enzyme E₁ may have polypeptide sequence SEQ ID NO: 11 or a polypeptide sequence in which up to 25%, preferably up to 20%, particularly preferably up to 15% in particular up to 10, 9, 8, 7, 6, 5, 4, 3, 2, 1% of the amino acid radicals are modified compared to the reference sequence SEQ ID NO: 11 by deletion, insertion, substitution or a combination thereof and that still has at least 10%, particularly 50%, more particularly 80%, even more particularly more than 90% of the enzymatic activity of the enzyme having the reference sequence SEQ ID NO: 11, wherein enzymatic activity for an enzyme E₁ is understood as meaning the ability preferably to convert 3-hydroxyalkanoyl-CoA/ACP into 3-hydroxyalkanoyl-3-hydroxyalkanoyl-CoA/ACP and further to HAA.

In a further example, the enzyme E₁ may have polypeptide sequence SEQ ID NO: 15 or a polypeptide sequence in which up to 25%, preferably up to 20%, particularly preferably up to 15% in particular up to 10, 9, 8, 7, 6, 5, 4, 3, 2, 1% of the amino acid radicals are modified compared to the reference sequence SEQ ID NO: 15 by deletion, insertion, substitution or a combination thereof and that still has at least 10%, particularly 50%, more particularly 80%, even more particularly more than 90% of the enzymatic activity of the enzyme having the reference sequence SEQ ID NO: 15, wherein enzymatic activity for an enzyme E₁ is understood as meaning the ability preferably to convert 3-hydroxyalkanoyl-CoA/ACP into 3-hydroxyalkanoyl-3-hydroxyalkanoyl-CoA/ACP and further to HAA.

The enzyme E₂ may be capable of converting 3-hydroxyalkanoyl-3-hydroxyalkanoyl-CoA/ACP into β-D-glucopyranosyl-3-hydroxyalkanoyl-3-hydroxyalkanoate. In one example, the enzyme E₂ may be capable of converting HAA into β-D-glucopyranosyl-3-hydroxyalkanoyl-3-hydroxyalkanoate. In yet another example, the enzyme E₂ may be capable of converting 3-hydroxyalkanoyl-3-hydroxyalkanoyl-CoA/ACP and HAA into β-D-glucopyranosyl-3-hydroxyalkanoyl-3-hydroxyalkanoate. In all these examples, NDP-glucose may be present particularly to include the glucose moiety in General Formula II. The enzyme E₂ may be a glycosyltransferase (EC 2.4). In particular, the enzyme E₂ comprises SEQ ID NO: 2 or variant thereof. The term “variant”, as used herein, comprises amino acid or nucleic acid sequences, respectively, that are at least 60, 65, 70, 75, 80, 85, 90, 92, 94, 95, 96, 97, 98 or 99% identical to the reference amino acid or nucleic acid sequence, wherein preferably amino acids other than those essential for the function, for example the catalytic activity of a protein, or the fold or structure of a molecule are deleted, substituted or replaced by insertions or essential amino acids are replaced in a conservative manner to the effect that the biological activity of the reference sequence or a molecule derived therefrom is preserved. The state of the art comprises algorithms that may be used to align two given nucleic acid or amino acid sequences and to calculate the degree of identity, see Arthur Lesk (2008), Thompson et al., 1994, and Katoh et al., 2005. The term “variant” is used synonymously and interchangeably with the term “homologue”. Such variants may be prepared by introducing deletions, insertions or substitutions in amino acid or nucleic acid sequences as well as fusions comprising such macromolecules or variants thereof. In one example, the term “variant”, with regard to amino acid sequence, comprises, in addition to the above sequence identity, amino acid sequences that comprise one or more conservative amino acid changes with respect to the respective reference or wild type sequence or comprises nucleic acid sequences encoding amino acid sequences that comprise one or more conservative amino acid changes. In one example, the term “variant” of an amino acid sequence or nucleic acid sequence comprises, in addition to the above degree of sequence identity, any active portion and/or fragment of the amino acid sequence or nucleic acid sequence, respectively, or any nucleic acid sequence encoding an active portion and/or fragment of an amino acid sequence. The term “active portion”, as used herein, refers to an amino acid sequence or a nucleic acid sequence, which is less than the full length amino acid sequence or codes for less than the full length amino acid sequence, respectively, wherein the amino acid sequence or the amino acid sequence encoded, respectively retains at least some of its essential biological activity. For example an active portion and/or fragment of a protease may be capable of hydrolysing peptide bonds in polypeptides. The phrase “retains at least some of its essential biological activity”, as used herein, means that the amino acid sequence in question has a biological activity exceeding and distinct from the background activity and the kinetic parameters characterising said activity, more specifically k_{cat} and K_{M}, are preferably within 3, 2, or 1 order of magnitude of the values displayed by the reference molecule with respect to a specific substrate. Similarly, the term “variant” of a nucleic acid comprises nucleic acids the complementary strand of which hybridises, preferably under stringent conditions, to the reference or wild type nucleic acid. In one example, the variant of SEQ ID: 4 may have 60% sequence identity to SEQ ID NO:4.

In one example, the enzyme E₂ may have polypeptide sequence SEQ ID NO: 2 or a polypeptide sequence in which up to 25%, particularly up to 20%, more particularly up to 15% in particular up to 10, 9, 8, 7, 6, 5, 4, 3, 2, 1% of the amino acid radicals are modified compared to the reference sequence SEQ ID NO: 2 by deletion, insertion, substitution or a combination thereof and that still has at least 10%, particularly 50%, more particularly 80%, even more particularly more than 90% of the enzymatic activity of the enzyme having the reference sequence SEQ ID NO: 2, wherein enzymatic activity for an enzyme E₂ is understood as meaning the ability particularly to convert 3-hydroxyalkanoyl-3-hydroxyalkanoyl-CoA/ACP or 3-(3-hydroxyalkanoyloxy)alkanoic acid (HAA) into β-D-glucopyranosyl-3-hydroxyalkanoyl-3-hydroxyalkanoate. NDP-glucose may be present in this conversion.

In another example, the enzyme E₂ may have polypeptide sequence SEQ ID NO: 8 or a polypeptide sequence in which up to 25%, particularly up to 20%, more particularly up to 15% in particular up to 10, 9, 8, 7, 6, 5, 4, 3, 2, 1% of the amino acid radicals are modified compared to the reference sequence SEQ ID NO: 8 by deletion, insertion, substitution or a combination thereof and that still has at least 10%, particularly 50%, more particularly 80%, even more particularly more than 90% of the enzymatic activity of the enzyme having the reference sequence SEQ ID NO: 8, wherein enzymatic activity for an enzyme E₂ is understood as meaning the ability particularly to convert 3-hydroxyalkanoyl-3-hydroxyalkanoyl-CoA/ACP or 3-(3-hydroxyalkanoyloxy)alkanoic acid (HAA) into β-D-glucopyranosyl-3-hydroxyalkanoyl-3-hydroxyalkanoate. NDP-glucose may be present in this conversion.

In one example, the enzyme E₂ may have polypeptide sequence SEQ ID NO: 12 or a polypeptide sequence in which up to 25%, particularly up to 20%, more particularly up to 15% in particular up to 10, 9, 8, 7, 6, 5, 4, 3, 2, 1% of the amino acid radicals are modified compared to the reference sequence SEQ ID NO: 12 by deletion, insertion, substitution or a combination thereof and that still has at least 10%, particularly 50%, more particularly 80%, even more particularly more than 90% of the enzymatic activity of the enzyme having the reference sequence SEQ ID NO: 12, wherein enzymatic activity for an enzyme E₂ is understood as meaning the ability particularly to convert 3-hydroxyalkanoyl-3-hydroxyalkanoyl-CoA/ACP or 3-(3-hydroxyalkanoyloxy)alkanoic acid (HAA) into β-D-glucopyranosyl-3-hydroxyalkanoyl-3-hydroxyalkanoate. NDP-glucose may be present in this conversion.

In particular, the use of SEQ ID NO: 2, SEQ ID NO: 8, or SEQ ID NO: 12 (i.e. from Pantoea spec.) as E₂ compared to the use of E₂ from other organisms, e.g. Serratia rubidaea may lead to production of higher amounts of rubiwettins.

In one example, the cell according to any aspect of the present invention is genetically modified to increase the heterologous expression relative to the wild-type cell of:
Enzyme E₁ a 3-(3-hydroxyalkanoyloxy)alkanoic acid (HAA) synthase comprising SEQ ID NO: 1, or a variant thereof, wherein the variant comprises 60% sequence identity to SEQ ID NO: 1; and
Enzyme E₂ a glycosyltransferase (EC 2.4) comprising SEQ ID NO: 2, or a variant thereof, wherein the variant comprises 60% sequence identity to SEQ ID NO:2.

In another example, the cell according to any aspect of the present invention is genetically modified to increase the heterologous expression relative to the wild-type cell of:
Enzyme E₁ a 3-(3-hydroxyalkanoyloxy)alkanoic acid (HAA) synthase comprising SEQ ID NO: 7, or a variant thereof, wherein the variant comprises 60% sequence identity to SEQ ID NO: 7; and
Enzyme E₂ a glycosyltransferase (EC 2.4) comprising SEQ ID NO: 8, or a variant thereof, wherein the variant comprises 60% sequence identity to SEQ ID NO:8.

In a further example, the cell according to any aspect of the present invention is genetically modified to increase the heterologous expression relative to the wild-type cell of:
Enzyme E₁ a 3-(3-hydroxyalkanoyloxy)alkanoic acid (HAA) synthase comprising SEQ ID NO: 11, or a variant thereof, wherein the variant comprises 60% sequence identity to SEQ ID NO: 11; and
Enzyme E₂ a glycosyltransferase (EC 2.4) comprising SEQ ID NO: 12, or a variant thereof, wherein the variant comprises 60% sequence identity to SEQ ID NO:12.

In one example, the cell according to any aspect of the present invention is genetically modified to increase the heterologous expression relative to the wild-type cell of:
Enzyme E₁ a 3-(3-hydroxyalkanoyloxy)alkanoic acid (HAA) synthase comprising SEQ ID NO: 15, or a variant thereof, wherein the variant comprises 60% sequence identity to SEQ ID NO: 15; and
Enzyme E₂ a glycosyltransferase (EC 2.4) comprising SEQ ID NO: 2, or a variant thereof, wherein the variant comprises 60% sequence identity to SEQ ID NO:2.

In another example, the cell according to any aspect of the present invention is genetically modified to increase the heterologous expression relative to the wild-type cell of:
Enzyme E₁ a 3-(3-hydroxyalkanoyloxy)alkanoic acid (HAA) synthase comprising SEQ ID NO: 15, or a variant thereof, wherein the variant comprises 60% sequence identity to SEQ ID NO: 15; and
Enzyme E₂ a glycosyltransferase (EC 2.4) comprising SEQ ID NO: 8, or a variant thereof, wherein the variant comprises 60% sequence identity to SEQ ID NO:8.

In another example, the cell according to any aspect of the present invention is genetically modified to increase the heterologous expression relative to the wild-type cell of:
Enzyme E₁ a 3-(3-hydroxyalkanoyloxy)alkanoic acid (HAA) synthase comprising SEQ ID NO: 15, or a variant thereof, wherein the variant comprises 60% sequence identity to SEQ ID NO: 15; and
Enzyme E₂ a glycosyltransferase (EC 2.4) comprising SEQ ID NO: 12, or a variant thereof, wherein the variant comprises 60% sequence identity to SEQ ID NO:12.

The lipid of general formula II may also be called a glucolipid. The length of the R¹ and R² group can be of varying lengths. In particular, R¹ and R² may be independently selected from the group consisting of saturated and unsaturated alkyls. More in particular, R¹ and/or R² may be a saturated or unsaturated alkyl group with 5 to 13 carbon atoms. Even more in particular, R¹ and R² may be a saturated or monounsaturated alkyl group with 5 to 13 carbon atoms. The R¹ and R² alkyl groups may comprise 5 to 13 carbon atoms, 7 to 13 carbon atoms, 9 to 13 carbon atoms, 5 to 11 carbon atoms, 5 to 9 carbon atoms and the like. In the examples where R¹ and/or R² alkyl group is a saturated alkyl group, the alkyl group may comprising 5, 6, 7, 8, 9, 10, 11, 12 or 13 carbon atoms. In the examples where R¹ and/or R² alkyl group is an unsaturated alkyl group, the alkyl group may be a monounsaturated alkyl comprising 5:1, 6:1, 7:1, 8:1, 9:1, 10:1, 11:1, 12:1, or 13:1 carbon atoms. Even more in particular, the R¹ and/or R² alkyl group is a saturated alkyl group with 5 to 13 carbon atoms. The cell according to any aspect of the present invention may be able to produce a mixture of rubiwettins with varying R¹ and R² groups. In one example, the lipid of general formula II produced according to any aspect of the present invention may be a rubiwettin RG1 (CAS-Nr. 129039-46-9). The rubiwettin RG1 may also be called a glucolipid named β-D-Glucopyranosyl-3-(3′-hydroxytetradecanoyloxy)decanoate or β-Glucopyranosyl-3-(3′-hydroxytetradecanoyloxy)decanoate.

The cell according to any aspect of the present invention may produce a further lipid with general formula I from a carbon substrate,

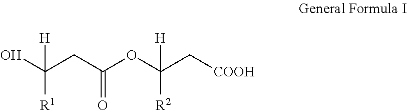

wherein R¹ and R² independently of one another is an identical or different alkyl group with 5 to 13 carbon atoms. In particular, the alkyl group may be saturated or unsaturated. More in particular, the alkyl group of R¹ and/or R² may be a saturated alkyl radical. Even more in particular, R¹ and/or R² may be selected from the group consisting of pentyl, heptyl, nonyl, undecyl, tridecyl and (CH₂)ₙ—CH₃ with n=4-12.

The lipid of general formula I may have R¹ and R² groups of varying lengths. In particular, R¹ and R² may be independently selected from the group consisting of saturated and unsaturated alkyls. More in particular, R¹ and/or R² may be a saturated or unsaturated alkyl group with 5 to 13 carbon atoms. Even more in particular, R¹ and R² may be a saturated or monounsaturated alkyl group with 5 to 13 carbon atoms. The R¹ and R² alkyl groups may comprise 5 to 13 carbon atoms, 7 to 13 carbon atoms, 9 to 13 carbon atoms, 5 to 11 carbon atoms, 5 to 9 carbon atoms and the like. In the examples where R¹ and/or R² alkyl group is an unsaturated alkyl group, the alkyl group may be a monounsaturated alkyl comprising 5:1, 6:1, 7:1, 8:1, 9:1, 10:1, 11:1, 12:1, or 13:1 carbon atoms. In the examples where R¹ and/or R² alkyl group is a saturated alkyl group, the alkyl group may comprising 5, 6, 7, 8, 9, 10, 11, 12 or 13 carbon atoms. The cell according to any aspect of the present invention may be able to produce a mixture of lipids with varying R¹ and R² groups. In particular, the lipid with formula I may also be called a rubiwettin R1 (CAS-Nr. 129039-45-8). In particular, the lipid is a mixture of 3-(3′-hydroxytetradecanoyloxy)tetradecanoate, 3-(3′-hydroxydecanoyloxy)decanoate, 3-(3′-hydroxyhexadecenoyloxy)hexadecenoate, 3-(3′-hydroxytetradecanoyloxy)decanoate, 3-(3′-hydroxyhexadecenoyloxy)decanoate, 3-(3′-hydroxyhexadecenoyloxy)tetradecanoate and minor molecular isomers.

Surprisingly, it could be shown that recombinant cells according to any aspect of the present invention with increased expression of E₂ and/or E₁ are able to produce increased amounts of lipids with the formulas II and/or I compared to the wildtype of the cell. The cells according to any aspect of the present invention may thus allow for high selective production of rubiwettins RG1 with reduced production of undesirable intermediates like dimers of p-hydroxy fatty acids.

In particular, the use of SEQ-IDs 1, 7, and 11 from Pantoea spec. as E₁ instead of sequences of E₁ from Pseudomonas aeruginosa leads to a shift of rubiwettin species from C10:0-C12:1 and C10:0-C12:0 towards C8:0-C10:0. The rubiwettin species with shorter 3-hydroxyfatty acid chain length may have different application profiles than the rubiwettin species with longer 3-hydroxyfatty acid chain length.

The phrase “increased heterologous expression of an enzyme”, as used herein is to be understood as increased intracellular activity. Basically, an increase in enzymatic activity can be achieved by increasing the copy number of the gene sequence or gene sequences that code for the enzyme, using a strong promoter or employing a gene or allele that codes for a corresponding enzyme with increased activity and optionally by combining these measures. Genetically modified cells used in the method according to the invention are for example produced by transformation, transduction, conjugation or a combination of these methods with a vector that contains the desired gene, an allele of this gene or parts thereof and a vector that makes expression of the gene possible. Heterologous expression is in particular achieved by integration of the gene or of the alleles in the chromosome of the cell or an extrachromosomally replicating vector. In particular, an increase in an activity of an enzyme relative to the wild type cell may be a 10, 15, 20, 25, 30, 35, 40, 45, 50, 55, 60, 65, 70, 75, 80, 85, 90, 95 or 100% more than the wild type cell.

A skilled person would be able to use any method known in the art to genetically modify a cell. Whether or not a nucleic acid molecule, polypeptide, more specifically an enzyme used according to any aspect of the present invention, is recombinant or not has not necessarily implications for the level of its expression. However, in one example one or more recombinant nucleic acid molecules, polypeptides or enzymes used according to any aspect of the present invention may be overexpressed. The term “overexpressed”, as used herein, means that the respective polypeptide encoded or expressed is expressed at a level higher or at higher activity than would normally be found in the cell under identical conditions in the absence of genetic modifications carried out to increase the expression, for example in the respective wild type cell. The person skilled in the art is familiar with numerous ways to bring about overexpression. For example, the nucleic acid molecule to be overexpressed or encoding the polypeptide or enzyme to be overexpressed may be placed under the control of a strong inducible promoter such as the lac promoter. The state of the art describes standard plasmids that may be used for this purpose, for example the pET system of vectors exemplified by pET-3a (commercially available from Novagen). Whether or not a nucleic acid or polypeptide is overexpressed may be determined by way of quantitative PCR reaction in the case of a nucleic acid molecule, SDS polyacrylamide electrophoreses, Western blotting or comparative activity assays in the case of a polypeptide. Genetic modifications may be directed to transcriptional, translational, and/or post-translational modifications that result in a change of enzyme activity and/or selectivity under selected and/or identified culture conditions. Thus, in various examples of the present invention, to function more efficiently, a microorganism may comprise one or more gene deletions. Gene deletions may be accomplished by mutational gene deletion approaches, and/or starting with a mutant strain having reduced or no expression of one or more of these enzymes, and/or other methods known to those skilled in the art.

DE-A-100 31 999 gives a general survey of the possibilities for increasing the enzyme activity in cells as exemplified by pyruvate carboxylase, which is inserted hereby as a reference and whose disclosure content with respect to the possibilities for increasing the enzyme activity in cells forms a part of the disclosure of the present invention.

The expression of the above and all subsequently mentioned enzymes or genes is detectable with the aid of 1- and 2-dimensional protein gel separation and subsequent optical identification of the protein concentration in the gel using appropriate analytical software. If the increase in an enzyme activity is based exclusively on an increase in the expression of the corresponding gene, the quantification of the increase in the enzyme activity can be determined in a simple manner by a comparison of the 1- or 2-dimensional protein separations between wild-type and genetically modified cell. A customary method for the preparation of the protein gels in the case of coryneforme bacteria and for the identification of the proteins is the procedure described by Hermann et al. (Electrophoresis, 22: 1712.23 (2001)). The protein concentration can likewise be analyzed by Western Blot hybridization using an antibody specific for the protein to be detected (Sambrook et al., Molecular Cloning: a laboratory manual, 2nd Ed. Cold Spring Harbor Laboratory Press, Cold Spring Harbor, N.Y. USA, 1989) and subsequent optical analysis using appropriate software for the concentration determination (Lohaus and Meyer (1989) Biospektrum, 5: 32-39; Lottspeich (1999) Angewandte Chemie 111: 2630-2647). The activity of DNA-binding proteins can be measured by means of DNA band shift assays (also called gel retardation) (Wilson et al. (2001) Journal of Bacteriology, 183: 2151-2155). The action of DNA-binding proteins on the expression of other genes can be detected by various well-described methods of the reporter gene assay (Sambrook et al., Molecular Cloning: a laboratory manual, 2nd Ed. Cold Spring Harbor Laboratory Press, Cold Spring Harbor, N.Y. USA, 1989). The intracellular enzymatic activities can be determined according to various described methods (Donahue et al. (2000) Journal of Bacteriology 182 (19): 5624-5627; Ray et al. (2000) Journal of Bacteriology 182 (8): 2277-2284; Freedberg et al. (1973) Journal of Bacteriology 115 (3): 816-823). If in the following embodiments no practical methods are indicated for the determination of the activity of a certain enzyme, the determination of the increase in the enzyme activity and also the determination of the decrease of an enzyme activity preferably take place by means of the methods described in Hermann et al., Electophoresis, 22: 1712-23 (2001), Lohaus et al., Biospektrum 5 32-39 (1998), Lottspeich, Angewandte Chemie 111: 2630-2647 (1999) and Wilson et al., Journal of Bacteriology 183: 2151-2155 (2001).

If the increase in the enzyme activity is accomplished by mutation of the endogenous gene, such mutations can be randomly produced either by conventional methods, such as, for example, by UV irradiation or by mutagenic chemicals, or selectively by means of genetic engineering methods such as deletion(s), insertion(s) and/or nucleotide exchange(s). Modified cells are obtained by these mutations. Particularly preferred mutants of enzymes are in particular also those enzymes that are no longer feedback-, product- or substrate-inhibitable or are so to a reduced degree at least in comparison to the wild-type enzyme.

If the increase in the enzyme activity is accomplished by increase in the synthesis of an enzyme, the copy number of the corresponding genes is increased or the promoter and regulation region or the ribosome binding site, which is situated upstream of the structural gene, is mutated. Expression cassettes, which are incorporated upstream of the structural gene, act in the same manner. It is additionally possible, by means of inducible promoters, to increase the expression at any desired point in time. In addition, however, also “enhancers” can be assigned to the enzyme gene as regulatory sequences, which likewise bring about increased gene expression by means of an improved interaction between RNA polymerase and DNA. As a result of measures for the prolongation of the lifetime of the mRNA, the expression is likewise improved. Furthermore, by prevention of the degradation of the enzyme protein the enzyme activity is likewise increased. The genes or gene constructs are present here either in plasmids having a different copy number or are integrated and amplified in the chromosome. Alternatively, an overexpression of the genes concerned can furthermore be achieved by modification of the media composition and culture management. The person skilled in the art finds directions for this, inter alia, in Martin et al. (Bio/Technology 5, 137-146 (1987)), in Guerrero et al. (Genes 138, 35-41 (1994)), Tsuchiya and Morinaga (Bio/Technology 6, 428-430 (1988)), in Eikmanns et al. (Genes 102, 93-98 (1991)), in EP-A-0 472 869, in U.S. Pat. No. 4,601,893, in Schwarzer and Puhler (Bio/Technology 9, 84-87 (1991)), in Reinscheid et al. (Applied and Environmental Microbiology 60, 126-132 (1994)), in LaBarre et al. (Journal of Bacteriology 175, 1001-1007 (1993)), in WO-A-96/15246, in Malumbres et al. (Genes 134, 15-24 (1993)), in JP-A-10-229891, in Jensen and Hammer (Biotechnology and Bioengineering 58, 191-195 (1998)) and in known textbooks of genetics and molecular biology. The measures described above likewise lead, like the mutations, to genetically modified cells.

Episomal plasmids, for example, are employed for increasing the expression of the respective genes. Suitable plasmids or vectors are in principle all embodiments available for this purpose to the person skilled in the art. Such plasmids and vectors can be taken, for example, from the brochures of the companies Novagen, Promega, New England Biolabs, Clontech or Gibco BRL. Further preferred plasmids and vectors can be found in: Glover, D. M. (1985) DNA cloning: a practical approach, Vol. I-III, IRL Press Ltd., Oxford; Rodriguez, R. L. and Denhardt, D. T (eds) (1988) Vectors: a survey of molecular cloning vectors and their uses, 179-204, Butterworth, Stoneham; Goeddel, D. V. (1990) Systems for heterologous gene expression, Methods Enzymol. 185, 3-7; Sambrook, J.; Fritsch, E. F. and Maniatis, T. (1989), Molecular cloning: a laboratory manual, 2nd ed., Cold Spring Harbor Laboratory Press, New York.

The plasmid vector, which contains the gene to be amplified, is then converted to the desired strain by conjugation or transformation. The method of conjugation is described, for example, in Schäfer et al., Applied and Environmental Microbiology 60: 756-759 (1994). Methods for transformation are described, for example, in Thierbach et al., Applied Microbiology and Biotechnology 29: 356-362 (1988), Dunican and Shivnan, Bio/Technology 7: 1067-1070 (1989) and Tauch et al., FEMS Microbiology Let-ters 123: 343-347 (1994). After homologous recombination by means of a “cross-over” event, the resulting strain contains at least two copies of the gene concerned.

According to any aspect of the present invention, the cell may be genetically modified so that in a defined time interval, within 2 hours, in particular within 8 hours or 24 hours, it forms at least twice, especially at least 10 times, at least 100 times, at least 1000 times or at least 10000 times more lipids of the general Formula I or II than the wild-type cell. The increase in product formation can be determined for example by cultivating the cell according to any aspect of the present invention and the wild-type cell each separately under the same conditions (same cell density, same nutrient medium, same culture conditions) for a specified time interval in a suitable nutrient medium and then determining the amount of target product (lipid with general formula II or I) in the nutrient medium.

Changes of amino acid residues of a given polypeptide sequence, which lead to no significant changes in the properties and function of the given polypeptide, are known to the person skilled in the art. Thus, for example, “conserved amino acids” can be mutually exchanged; examples of such suitable amino acid substitutions are: Ala for Ser; Arg for Lys; Asn for Gln or His; Asp for Glu; Cys for Ser; Gln for Asn; Glu for Asp; Gly for Pro; His for Asn or Gln; Ile for Leu or Val; Leu for Met or Val; Lys for Arg or Gln or Glu; Met for Leu or lie; Phe for Met or Leu or Tyr; Ser for Thr; Thr for Ser; Trp for Tyr; Tyr for Trp or Phe; Val for Ile or Leu. It is likewise known that changes, particularly at the N- or C-terminus of a polypeptide, in the form of, for example, amino acid insertions or deletions often exert no significant influence on the function of the polypeptide.

The activity of an enzyme can be determined by disrupting cells which contain this activity in a manner known to the person skilled in the art, for example with the aid of a ball mill, a French press or of an ultrasonic disintegrator and subsequently separating off cells, cell debris and disruption aids, such as, for example, glass beads, by centrifugation for 10 minutes at 13,000 rpm and 4° C. Using the resulting cell-free crude extract, enzyme assays with subsequent LC-ESI-MS detection of the products can then be carried out. Alternatively, the enzyme can be enriched in the manner known to the person skilled in the art by chromatographic methods (such as nickel-nitrilotriacetic acid affinity chromatography, streptavidin affinity chromatography, gel filtration chromatography or ion-exchange chromatography) or else purified to homogeneity.

In one example the method used to determine the activity of enzyme E₂ involves first disrupting cells which contain this activity (i.e. the cells according to any aspect of the present invention) in a manner known to the person skilled in the art, for example with the aid of a ball mill, a French press or an ultrasonic disintegrator and subsequently separating of cells, cell debris and disruption aids, such as, for example, glass beads, by centrifugation for 10 min at 16,100 g at 4° C. Using the resulting cell-free crude extract, enzyme assays with subsequent LC-ESI-MS detection of the products can be carried out. As an alternative, the enzyme can be enriched in the manner known to the person skilled in the art by chromatography methods (such as nickel/nitrilotriacetic acid affinity chromatography, streptavidin affinity chromatography, gel filtration chromatography or ion-exchange chromatography) or else purified to homogeneity. This sample may then be used to measure the activity of enzyme E₂. In particular, the activity of enzyme E₂ may be determined using a standard assay that may consists of 185 μl of 10 mM Tris-HCl (pH 7.5), 10 μl mM NDP-glucose and 50 μl of protein crude extract (about 1 mg of total protein) or purified protein in solution (5 μg of purified protein). The reaction may be started by the addition of 10 μl mM ethanolic solution of 3-hydroxytetradecanoyl-3-hydroxydecanoic acid or 3-hydroxyhexadecanoyl-3-hydroxydecanoic acid and incubated for 1 h at 30° C. with shaking (600 rpm). Subsequently, the reaction may be treated with 1 ml of acetone. Undissolved constituents, may be sedimented by centrifugation (16,100 g, 5 min RT) and the sample may be analyzed by means of LC-ESI-MS. The identification of the products may then take place by analysis of the corresponding mass traces and the MS2 spectra. This method may be used to measure the activity of E₂.

In another example, the method used to determine the activity of enzyme E₁ involves first disrupting cells which contain this activity (i.e. the cells according to any aspect of the present invention) in a manner known to the person skilled in the art and subsequently separating of cells, cell debris and disruption aids, such as, for example, glass beads, by centrifugation for 10 min at 16,100 g at 4° C. Using the resulting cell-free crude extract, enzyme assays with subsequent LC-ESI-MS detection of the products can be carried out. As an alternative, the enzyme can be enriched in the manner known to the person skilled in the art by chromatography methods (such as nickel/nitrilotriacetic acid affinity chromatography, streptavidin affinity chromatography, gel filtration chromatography or ion-exchange chromatography) or else purified to homogeneity. This sample may then be used to measure the activity of enzyme E₁. In particular, the activity of enzyme E₁ may be determined using a standard assay which may contain 100 μM E. coli ACP, 1 mM β-mercaptoethanol, 200 μM malonyl-coenzyme A, 40 μM octanoyl-coenzyme A and 40 μM dodecanoyl-coenzyme A or 40 μM octanoyl-coenzyme A and 40 mM tetradecanoyl-coenzyme A, 100 μM NADPH, 2 μg of E. coli FabD, 2 μg of Mycobacterium tuberculosis FabH, 1 μg of E. coli FabG, 0.1 M sodium phosphate buffer (pH 7.0), and 5 μg of enzyme E₁ in a final volume of 120 μl. ACP, β-mercaptoethanol and sodium phosphate buffer may be preincubated for 30 min at 37° C. to reduce the ACP completely. The reaction may be started by addition of enzyme E₁. The reactions may be stopped using 2 ml of water, which has been acidified with HCl to pH 2.0, and subsequently extracted twice with 2 ml of chloroform/methanol (2:1 (v:v)). Phase separation may take place by centrifugation (16,100 g, 5 min, RT). The lower organic phase may be removed, evaporated completely in the vacuum centrifuge and the sediment may be taken up in 50 μl of methanol. Undissolved constituents, may be sedimented by centrifugation (16,100 g, 5 min RT) and the sample may be analyzed by means of LC-ESI-MS. The identification of the products may take place by analysis of the corresponding mass traces and the MS2 spectra.

The enzyme used according to any aspect of the present invention may be recombinant. The term “recombinant” as used herein, refers to a molecule or is encoded by such a molecule, particularly a polypeptide or nucleic acid that, as such, does not occur naturally but is the result of genetic engineering or refers to a cell that comprises a recombinant molecule. For example, a nucleic acid molecule is recombinant if it comprises a promoter functionally linked to a sequence encoding a catalytically active polypeptide and the promoter has been engineered such that the catalytically active polypeptide is overexpressed relative to the level of the polypeptide in the corresponding wild type cell that comprises the original unaltered nucleic acid molecule.

The cell used according to any aspect of the present invention may also be a non-pathogenic cell. A non-pathogenic cell refers to a cell that does not cause disease, harm or death to another organism. The cells according to any aspect of the present invention may any non-pathogenic prokaryote or eukaryote. These can be mammalian cells (such as, for example, cells from man), plant cells or microorganisms such as yeasts, fungi or bacteria, wherein microorganisms in particular bacteria and yeasts are preferred.

Suitable bacteria, yeasts or fungi are in particular those bacteria, yeasts or fungi that are deposited in the Deutsche Sammlung von Mikroorganismen und Zellkulturen (German Collection of Microorganisms and Cell Cultures) GmbH (DSMZ), Brunswick, Germany, as bacterial, yeast or fungal strains. Bacteria suitable according to the invention belong to the genera that are listed under:
http://www.dsmz.de/species/bacteria.htm,
   yeasts suitable according to the invention belong to those genera that are listed under:
http://www.dsmz.de/species/yeasts.htm
   and fungi suitable according to the invention are those that are listed under:
http://www.dsmz.de/species/fungi.htm.

In particular, the cells may be selected from the genera Aspergillus, Corynebacterium, Brevibacterium, Bacillus, Acinetobacter, Alcaligenes, Lactobacillus, Paracoccus, Lactococcus, Candida, Pichia, Hansenula, Kluyveromyces, Saccharomyces, Escherichia, Zymomonas, Yarrowia, Methylobacterium, Ralstonia, Pseudomonas, Rhodospirillum, Rhodobacter, Burkholderia, Clostridium and Cupriavidus. More in particular, the cells may be selected from the group consisting of Aspergillus nidulans, Aspergillus niger, Alcaligenes latus, Bacillus megaterium, Bacillus subtilis, Brevibacterium flavum, Brevibacterium lactofermentum, Burkholderia andropogonis, B. brasilensis, B. caledonica, B. caribensis, B. caryophylli, B. fungorum, B. gladioli, B. glathei, B. glumae, B. graminis, B. hospita, B. kururiensis, B. phenazinium, B. phymatum, B. phytofirmans, B. plantarii, B. sacchari, B. singaporensis, B. sordidicola, B. terricola, B. tropica, B. tuberum, B. ubonensis, B. unamae, B. xenovorans, B. anthina, B. pyrrocinia, B. thailandensis, Candida blankii, Candida rugosa, Corynebacterium glutamicum, Corynebacterium efficiens, Escherichia coli, Hansenula polymorpha, Kluveromyces lactis, Methylobacterium extorquens, Paracoccus versutus, Pseudomonas argentinensis, P. borbori, P. citronellolis, P. flavescens, P. mendocina, P. nitroreducens, P. oleovorans, P. pseudoalcaligenes, P. resinovorans, P. straminea, P. aurantiaca, P. aureofaciens, P. chlororaphis, P. fragi, P. lundensis, P. taetrolens, P. antarctica, P. azotoformans, ‘P. blatchfordae’, P. brassicacearum, P. brenneri, P. cedrina, P. corrugata, P. fluorescens, P. gessardii, P. libanensis, P. mandelii, P. marginalis, P. mediterranea, P. meridiana, P. migulae, P. mucidolens, P. orientalis, P. panacis, P. proteolytica, P. rhodesiae, P. synxantha, P. thivervalensis, P. tolaasii, P. veronii, P. denitrificans, P. pertucinogena, P. cremoricolorata, P. fulva, P. monteilii, P. mosselii, P. parafulva, P. putida, P. balearica, P. stutzeri, P. amygdali, P. avellanae, P. caricapapayae, P. cichorii, P. coronafaciens, P. ficuserectae, ‘P. helianthi’, P. meliae, P. savastanoi, P. syringae, P. tomato, P. viridiflava, P. abietaniphila, P. acidophila, P. agarici, P. alcaliphila, P. alkanolytica, P. amyloderamosa, P. asplenii, P. azotifigens, P. cannabina, P. coenobios, P. congelans, P. costantinii, P. cruciviae, P. delhiensis, P. excibis, P. extremorientalis, P. frederiksbergensis, P. fuscovaginae, P. gelidicola, P. grimontii, P. indica, P. jessenii, P. jinjuensis, P. kilonensis, P. knackmussii, P. koreensis, P. lini, P. lutea, P. moraviensis, P. otitidis, P. pachastrellae, P. palleroniana, P. papaveris, P. peli, P. perolens, P. poae, P. pohangensis, P. psychrophila, P. psychrotolerans, P. rathonis, P. reptilivora, P. resiniphila, P. rhizosphaerae, P. rubescens, P. salomonii, P. segitis, P. septica, P. simiae, P. suis, P. thermotolerans, P. aeruginosa, P. tremae, P. trivialis, P. turbinellae, P. tuticorinensis, P. umsongensis, P. vancouverensis, P. vranovensis, P. xanthomarina, Ralstonia eutropha, Rhodospirillum rubrum, Rhodobacter sphaeroides, Saccharomyces cerevisiae, Yarrowia lipolytica and Zymomonas mobile. More in particular, the cell may be a bacterial cell selected from the group consisting of Acinetobacter sp., Bacillus sp., Brevibacterium sp., Burkholderia sp., Chlorella sp., Clostridium sp., Corynebacterium sp., Cyanobakterien, Escherichia sp., Pseudomonas sp., Klebsiella sp., Salmonella sp., Rhizobium sp., Saccharomyces sp., Pichia sp., and Nostoc sp. Even more in particular, the cell may be selected from the group consisting of Bacillus subtilis, Burkholderia thailandensis, Corynebacterium glutamicum, E. coli, Klebsiella oxytoca, Pseudomonas fluorescens, Pseudomonas putida, Pseudomonas stutzeri. Rhizobium meliloti. Saccharomyces cerevisiae and Pichia pastoris.

In one example, the cell according to any aspect of the present invention may be a cell that is genetically modified to increase the expression of
enzyme E₂ comprises SEQ ID NO: 2, and
enzyme E₁ comprises SEQ ID NO: 1 or variant thereof.

In another example, the cell according to any aspect of the present invention may be a cell that is genetically modified to increase the expression of:
enzyme E₁ comprising SEQ ID NO: 1 or variant thereof and enzyme E₂ comprising SEQ ID NO: 8 or variant thereof, or
enzyme E₁ comprising SEQ ID NO: 1 or variant thereof and enzyme E₂ comprising SEQ ID NO: 12 or variant thereof, or
enzyme E₁ comprising SEQ ID NO: 7 or variant thereof and enzyme E₂ comprising SEQ ID NO: 2 or variant thereof, or
enzyme E₁ comprising SEQ ID NO: 7 or variant thereof and enzyme E₂ comprising SEQ ID NO: 8 or variant thereof, or
enzyme E₁ comprising SEQ ID NO: 7 or variant thereof and enzyme E₂ comprising SEQ ID NO: 12 or variant thereof, or
enzyme E₁ comprising SEQ ID NO: 11 or variant thereof and enzyme E₂ comprising SEQ ID NO: 2 or variant thereof, or
enzyme E₁ comprising SEQ ID NO: 11 or variant thereof and enzyme E₂ comprising SEQ ID NO: 8 or variant thereof, or
enzyme E₁ comprising SEQ ID NO: 11 or variant thereof and enzyme E₂ comprising SEQ ID NO: 12 or variant thereof, or
enzyme E₁ comprising SEQ ID NO: 15 or variant thereof and enzyme E₂ comprising SEQ ID NO: 2 or variant thereof,
enzyme E₁ comprising SEQ ID NO: 15 or variant thereof and enzyme E₂ comprising SEQ ID NO: 8 or variant thereof, or
enzyme E₁ comprising SEQ ID NO: 15 or variant thereof and enzyme E₂ comprising SEQ ID NO: 12 or variant thereof.

The cells according to any aspect of the present invention may be used to produce a lipid according to General formula I and/or II from a carbon substrate:

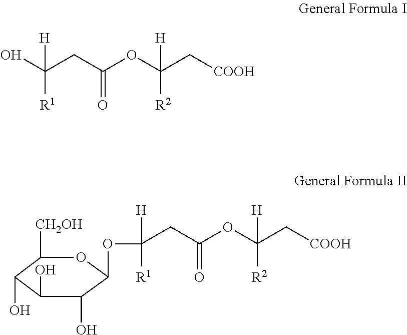

wherein R¹ and R² independently of one another in General Formula I or II is an identical or different alkyl group with 5 to 13 carbon atoms. In particular, the alkyl group may be saturated or unsaturated. More in particular, the alkyl group of R¹ and/or R² may be a saturated alkyl radical. Even more in particular, R¹ and/or R² may be selected from the group consisting of pentyl, heptyl, nonyl, undecyl, tridecyl, and (CH₂)ₙ—CH₃ with n=4-12.

The lipids formed may be combination of lipids with general formula I and II with varying R group that may be produced during a single reaction.

The genetically modified cells according to the invention can be brought into contact with the nutrient medium continuously or discontinuously in the batch process (batch culture) or in the fed-batch process (feed process) or repeated fed-batch process (repetitive feed process) for the purpose of the production of the abovementioned products and thus cultured. A semi-continuous process is also conceivable, as is described in GB-A-1009370. A summary of known culturing methods are described in the textbook of Chmiel (“Bioprozesstechnik 1. Einführung in die Bioverfahrenstechnik” [Bioprocess Technology 1. Introduction to the Bioprocess Technique] (Gustav Fischer Verlag, Stuttgart, 1991)) or in the textbook of Storhas (“Bioreaktoren und periphere Einrichtungen” [Bioreactors and Peripheral Devices], Vieweg Verlag, Brunswick/Wiesbaden, 1994).

The culture medium to be used must satisfy in a suitable manner the demands of the respective strains. Descriptions of culture media of different yeast strains are contained, for example, in “Nonconventional yeast in biotechnology” (Ed. Klaus Wolf, Springer-Verlag Berlin, 1996).

The carbon source used as a substrate according to any aspect of the present invention may be selected from the group consisting of carbohydrates such as, for example, glucose, sucrose, arabinose, xylose, lactose, fructose, maltose, molasses, starch, cellulose and hemicellulose, vegetable and animal oils and fats such as, for example, soybean oil, safflower oil, peanut oil, hempseed oil, jatropha oil, coconut fat, calabash oil, linseed oil, corn oil, poppyseed oil, evening primrose oil, olive oil, palm kernel oil, palm oil, rapeseed oil, sesame oil, sunflower oil, grapeseed oil, walnut oil, wheat germ oil and coconut oil, fatty acids, such as, for example, caprylic acid, capric acid, lauric acid, myristic acid, palmitic acid, palmitoleic acid, stearic acid, arachidonic acid, behenic acid, oleic acid, linoleic acid, linolenic acid, gamma-linolenic acid and its methyl or ethyl ester as well as fatty acid mixtures, mono-, di- and triglycerides containing the fatty acids just mentioned, alcohols such as, for example, glycerol, ethanol and methanol, hydrocarbons such as methane, ethane, propane or butane carbon-containing gases and gas mixtures, such as CO, CO₂, synthesis or flue gas, amino acids such as L-glutamate or L-valine or organic acids such as, for example, acetic acid. These substances can be used individually or as a mixture. The use of carbohydrates, in particular of monosaccharides, oligosaccharides or polysaccharides, as the carbon source as is described in U.S. Pat. No. 6,01,494 and U.S. Pat. No. 6,136,576 as well as of hydrocarbons, in particular of alkanes, alkenes and alkynes. In particular, the carbon source may be selected from the group consisting of glucose, dextrose, sucrose, mannose, galactose, polysaccharides, such as cellulose or hemicelluloses, vegetal oils, animal fats, fatty acids, fatty acid esters, carbonaceous gases, alkanes, glycerol, acetate, ethanol and methanol. More in particular, the carbon source may be selected from the group consisting of glucose, sucrose, glycerol, vegetal oils, methane, ethane, and butane. It is a great advantage of the present invention that the cells according to the invention are able to form lipids with general formula I and/or II from the simplest carbon sources such as, for example, glucose, sucrose or glycerol, such that a provision of longer-chain C sources in the medium during the method according to any aspect of the present invention is not necessary. In particular, the carbon source may be selected from the group consisting of glucose, dextrose, sucrose, xylose, mannose, galactose, arabinose, monosaccharides, polysaccharides, cellulose, hemicelluloses, vegetal oils, animal fats, fatty acids, fatty acid esters, carbonaceous gases, alkanes, glycerol, acetate, ethanol and methanol.

According to another aspect of the present invention, there is provided a microbial cell for producing at least one lipid with general formula II from at least one carbon substrate,

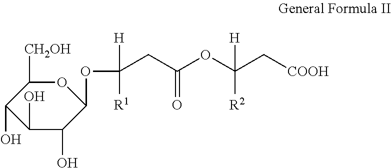

wherein R¹ and R² independently of one another comprises identical or different organic radicals each with 5 to 13 carbon atoms, wherein the cell is a non-pathogenic cell that is genetically modified to introduce a DNA sequence coding for:
   Enzyme E₁, a 3-(3-hydroxyalkanoyloxy)alkanoic acid (HAA) synthase, wherein the amino acid sequence of E₁ is selected from the group consisting of SEQ ID NO: 1, SEQ ID NO: 7, SEQ ID NO: 11, SEQ ID NO: 15, and a variant thereof, wherein the variant comprises 60% sequence identity to SEQ ID NO: 1, 7, 11 or 15; and
   Enzyme E₂, a glycosyltransferase (EC 2.4), wherein the amino acid sequence of E₂ is selected from the group consisting of SEQ ID NO: 2, SEQ ID NO: 8, SEQ ID NO: 11, and a variant thereof, wherein the variant comprises 60% sequence identity to SEQ ID NO: 2, 8 or 11.

According to a further aspect of the present invention there is provided a method of producing at least one lipid with general formula II:

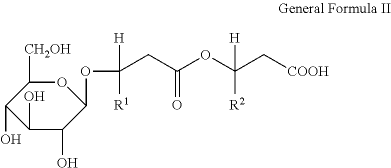

wherein R¹ and R² independently of one another is an identical or different alkyl group with 5 to 13 carbon atoms, and
wherein the method comprises a step of contacting at least one cell according to any aspect of the present invention with at least one carbon source.

In particular, the alkyl group of R¹ and/or R² may be saturated or unsaturated. More in particular, the alkyl group of R¹ and/or R² may be a saturated alkyl radical. Even more in particular, R¹ and/or R² may be selected from the group consisting of pentyl, heptyl, nonyl, undecyl, and (CH₂)ₙ—CH₃ with n=4-12.

The method according to any aspect of the present invention may also be used to produce a further lipid with general formula I from the carbon substrate,

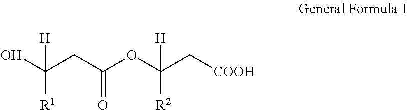

wherein R¹ and R² independently of one another is an identical or different alkyl group with 5 to 13 carbon atoms. In particular, the alkyl group of R¹ and/or R² may be saturated or unsaturated. More in particular, the alkyl group of R¹ and/or R² may be a saturated alkyl radical. Even more in particular, R¹ and/or R² may be selected from the group consisting of pentyl, heptyl, nonyl, undecyl, tridecyl, and (CH₂)ₙ—CH₃ with n=4-12.

The method according to any aspect of the present invention may be used to produce a mixture of lipids comprising the lipid in general formula I and II. In particular, the lipids of general formula I and II are produced in the ratio of 1:100, 1:90, 1:80, 1:70, 1:60, 1:50, 1:40, 1:30, 1:20, 1:10, 1:5, 1:4, 1:3, 1:2, 1:1, 2:1, 3:1, 4:1, 5:1, 10:1, 20:1, 30:1, 40:1, 50:1, 60:1, 70:1, 80:1, 90:1 or 100:1. More in particular lipids of formula I and II may have varying lengths of alkyls present simultaneously in the R subgroup.

According to yet a further aspect of the present invention, there is provided a use of the cell according to any aspect of the present invention for producing at least one lipid with general formula I and/or II:

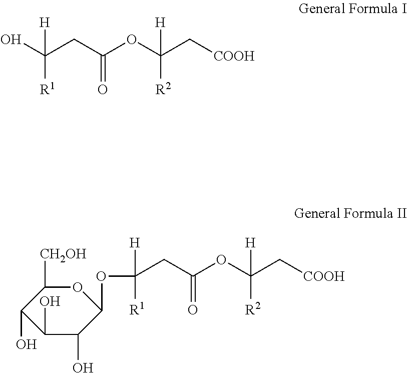

wherein R¹ and R² independently of one another is an identical or different alkyl group with 5 to 13 carbon atoms. In particular, the alkyl group of R¹ and/or R² may be saturated or unsaturated. More in particular, the alkyl group of R¹ and/or R² may be a saturated alkyl radical. Even more in particular, R¹ and/or R² may be selected from the group consisting of pentyl, heptyl, nonyl, undecyl, tridecyl and (CH₂)ₙ—CH₃ with n=4-12.

### EXAMPLES

The foregoing describes preferred embodiments, which, as will be understood by those skilled in the art, may be subject to variations or modifications in design, construction or operation without departing from the scope of the claims. These variations, for instance, are intended to be covered by the scope of the claims.

### Example 1

Construction of an Expression Vector for the Pantoea ananatis Strain PNA 99-7 NODE 2 Genes rbwAB

For the heterologous expression of the genes rbwA Pan997 as enzyme E₁ and rbwB Pan997 as enzyme E₂ from Pantoea ananatis strain PNA 99-7 NODE 2 the plasmid pACYC {PRhaRS}[rhaRS_Ec]{PRha}[rbwA_Pan997 rbwB_Pan997]{ter} was constructed. The synthetic operon consisting of rbwAB Pan997 (SEQ ID NO:3) which encode an 3-(3′-hydroxyalkanoyloxy)alkanoic acid (HAA) synthase (RbwA, SEQ ID NO:1) and a glucosyltransferase (RbwB, SEQ ID NO:2), respectively, was cloned under the control of the rhamnose inducible promoter Pᵣₕₐ into the vector pACYC {PRhaRS}[rhaRS_Ec]{PRha}[rhlA_Pa rbwB_Srub] as disclosed in the example of WO2019154984, which is based on pAYCY184 (New England Biolabs, Frankfurt/Main, Germany). Downstream of the synthetic operon a terminator sequence was located. The genes were amplified from synthetic DNA generated by Eurofins Genomics via PCR. The plasmid pACYC {PRhaRS}[rhaRS_Ec]{PRha}[rhlA_Pa rbwB_Srub] carries the P_{Rha} promoter cassette (SEQ ID NO:4) and the terminator sequence (SEQ ID NO:5), a p15A origin of replication for E. coli and a pVS1 origin of replication for the replication in P. putida KT2440. The pVS1 origin comes from the Pseudomonas plasmid pVS1 (Itoh Y, et al. Plasmid 1984, 11(3), 206-20). For amplification, the Q5® High-Fidelity 2× Master Mix from New England Biolabs (Frankfurt/Main, Germany) was used according to manufacturer's manual. In the next step the amplified fragment was cloned into the vector pACYC {PRhaRS}[rhaRS_Ec]{PRha}[rhlA_Pa rbwB_Srub] as disclosed in the example of WO2019154984 using the restriction sites ApaI/PspXI and NEBuilder® HiFi DNA Assembly Master Mix. The assembled product was transformed into NEB® 10-beta Electrocompetent E. coli cells (New England Biolabs, Frankfurt/Main, Germany). Procedure of PCR purification, cloning and transformation were carried out according to manufacturer's manual. The correct insertion of the target genes was checked by restriction analysis and the authenticity of the introduced DNA fragments was verified by DNA sequencing. The resulting plasmid was named pACYC {PRhaRS}[rhaRS_Ec]{PRha}[rbwA_Pan997 rbwB_Pan997]{ter} (SEQ ID NO:6).

The P. putida strain KT2440 was transformed with the plasmid pACYC {PRhaRS}[rhaRS_Ec]{PRha}[rbwA_Pan997 rbwB_Pan997]{ter} by means of electroporation (Iwasaki K, et al., Biosci. Biotech. Biochem. 1994. 58(5):851-854)) and plated onto LB-agar plates supplemented with kanamycin (50 μg/mL). Transformants were checked for the presence of the correct plasmid by plasmid preparation and analytical restriction analysis. The resulting strain was named BS-S-644 (P.putida KT2440+pACYC {PRhaRS}[rhaRS_Ec]{PRha}[rbwA_Pan997 rbwB_Pan997]{ter}).

### Example 2

Construction of an Expression Vector for the Pantoea sp. SJZ147 Genes rbwAB

For the heterologous expression of the genes rbwA_PanSJ as enzyme E₁ and rbwB_PanSJ as enzyme E₂ from Pantoea sp. SJZ147 the plasmid pACYC {PRhaRS}[rhaRS_Ec]{PRha}[rbwA_PanSJ rbwB_PanSJ]{ter}} was constructed. The synthetic operon consisting of rbwAB__PanSJ (SEQ ID NO:9) which encode an 3-(3′-hydroxyalkanoyloxy)alkanoic acid (HAA) synthase (RbwA, SEQ ID NO:7) and a glucosyltransferase (RbwB, SEQ ID NO:8), respectively, was cloned under the control of the rhamnose inducible promoter Pᵣₕₐ into the vector pACYC {PRhaRS}[rhaRS_Ec]{PRha}[rhlA_Pa rbwB_Srub] (WO2019154984) which is based on pAYCY184 (New England Biolabs, Frankfurt/Main, Germany). Downstream of the synthetic operon a terminator sequence is located. The genes were amplified from synthetic DNA generated by Eurofins Genomics via PCR. The plasmid pACYC {PRhaRS}[rhaRS_Ec]{PRha}[rhlA_Pa rbwB_Srub] carries the P_{Rha} promoter cassette (SEQ ID NO:4) and the terminator sequence (SEQ ID NO:5), a p15A origin of replication for E. coli and a pVS1 origin of replication for the replication in P. putida KT2440. The pVS1 origin comes from the Pseudomonas plasmid pVS1 (Itoh Y, et al. Plasmid 1984, 11(3), 206-20). For amplification the Q5® High-Fidelity 2× Master Mix from New England Biolabs (Frankfurt/Main, Germany) was used according to manufacturer's manual. In the next step the amplified fragment was cloned into the vector pACYC {PRhaRS}[rhaRS_Ec]{PRha}[rhlA_Pa rbwB_Srub] using the restriction sites ApaI/PspXI and NEBuilder® HiFi DNA Assembly Master Mix. The assembled product was transformed into NEB® 10-beta Electrocompetent E. coli cells (New England Biolabs, Frankfurt/Main, Germany). Procedure of PCR purification, cloning and transformation were carried out according to manufacturer's manual. The correct insertion of the target genes was checked by restriction analysis and the authenticity of the introduced DNA fragments was verified by DNA sequencing. The resulting plasmid was named pACYC {PRhaRS}[rhaRS_Ec]{PRha}[rbwA_PanSJ rbwB_PanSJ]{ter}} (SEQ ID NO:10).

The P. putida strain KT2440 was transformed with the plasmid pACYC {PRhaRS}[rhaRS_Ec]{PRha}[rbwA_PanSJ rbwB_PanSJ]{ter} by means of electroporation (Iwasaki K, et al., Biosci. Biotech. Biochem. 1994. 58(5):851-854)) and plated onto LB-agar plates supplemented with kanamycin (50 μg/mL). Transformants were checked for the presence of the correct plasmid by plasmid preparation and analytical restriction analysis. The resulting strain was named BS-S-645 (P.putida KT2440+pACYC {PRhaRS}[rhaRS_Ec]{PRha}[rbwA_PanSJ rbwB_PanSJ]{ter}).

### Example 3

Construction of an Expression Vector for the Pantoea stewartii Strain NS381 Genes rbwAB

For the heterologous expression of the genes rbwA_Pst381 as enzyme E₁ and rbwB_Pst381 as enzyme E₂ from Pantoea stewartii strain NS381 the plasmid pACYC {PRhaRS}[rhaRS_Ec]{PRha}[rbwA_Pst381 rbwB_Pst381]{ter} was constructed. The synthetic operon consisting of rbwAB_Pst381 (SEQ ID NO:13) which encode an 3-(3′-hydroxyalkanoyloxy)alkanoic acid (HAA) synthase (RbwA, SEQ ID NO:11) and a glucosyltransferase (RbwB, SEQ ID NO:12), respectively, was cloned under the control of the rhamnose inducible promoter Pᵣₕₐ into the vector pACYC {PRhaRS}[rhaRS_Ec]{PRha}[rhlA_Pa rbwB_Srub](WO2019154984), which is based on pAYCY184 (New England Biolabs, Frankfurt/Main, Germany). Downstream of the synthetic operon a terminator sequence is located. The genes were amplified from synthetic DNA generated by Eurofins Genomics via PCR. The plasmid pACYC {PRhaRS}[rhaRS_Ec]{PRha}[rhlA_Pa rbwB_Srub] carries the P_{Rha} promoter cassette (SEQ ID NO:4) and the terminator sequence (SEQ ID NO:5), a p15A origin of replication for E. coli and a pVS1 origin of replication for the replication in P. putida KT2440. The pVS1 origin comes from the Pseudomonas plasmid pVS1 (Itoh Y, et al. Plasmid 1984, 11(3), 206-20). For amplification the Q5® High-Fidelity 2× Master Mix from New England Biolabs (Frankfurt/Main, Germany) was used according to manufacturer's manual. In the next step the amplified fragment was cloned into the vector pACYC {PRhaRS}[rhaRS_Ec]{PRha}[rhlA_Pa rbwB_Srub] using the restriction sites ApaI/PspXI and NEBuilder® HiFi DNA Assembly Master Mix. The assembled product was transformed into NEB® 10-beta Electrocompetent E. coli cells (New England Biolabs, Frankfurt/Main, Germany). Procedure of PCR purification, cloning and transformation were carried out according to manufacturer's manual. The correct insertion of the target genes was checked by restriction analysis and the authenticity of the introduced DNA fragments was verified by DNA sequencing. The resulting plasmid was named pACYC {PRhaRS}[rhaRS_Ec]{PRha}[rbwA_Pst381 rbwB_Pst381]{ter} (SEQ ID NO:14).

The P. putida strain KT2440 was transformed with the plasmid pACYC {PRhaRS}[rhaRS_Ec]{PRha}[rbwA_Pst381 rbwB_Pst381]{ter} by means of electroporation (Iwasaki K, et al., Biosci. Biotech. Biochem. 1994. 58(5):851-854)) and plated onto LB-agar plates supplemented with kanamycin (50 μg/mL). Transformants were checked for the presence of the correct plasmid by plasmid preparation and analytical restriction analysis. The resulting strain was named BS-S-646 (P.putida KT2440+pACYC {PRhaRS}[rhaRS_Ec]{PRha}[rbwA_Pst381 rbwB_Pst381]{ter}).

### Example 4

Construction of an Expression Vector for the P. aeruginosa Gene Rh/a and Pantoea ananatis Strain PNA 99-7 NODE 2 Gene rbwB

For the heterologous expression of the genes rhlA_Pa as enzyme E₁ and rbwB_Pan997 as enzyme E₂ from Pantoea ananatis strain PNA 99-7 NODE_2 the plasmid pACYC {PRhaRS}[rhaRS_Ec]{PRha}[rhlA_Pa rbwB_Pan997]{ter} was constructed. The synthetic operon consisting of rhlA_Pa (SEQ ID NO:17) which encodes a 3-(3-hydroxyalkanoyloxy)alkanoic acid (HAA) synthase (RhlA, SEQ ID NO:16) and a glucosyltransferase (RbwB, SEQ ID NO:2), respectively, was cloned under the control of the rhamnose inducible promoter Pᵣₕₐ into the vector pACYC {PRhaRS}[rhaRS_Ec]{PRha}[rhlA_Pa rbwB_Srub] (WO2019154984), which is based on pAYCY184 (New England Biolabs, Frankfurt/Main, Germany). Downstream of the synthetic operon a terminator sequence is located. The gene rbwB_Pan997 was synthetically generated by Eurofins Genomics as DNA fragment. The plasmid pACYC {PRhaRS}[rhaRS_Ec]{PRha}[rhlA_Pa rbwB_Srub] carries the PRha promoter cassette (SEQ ID NO:4) and the terminator sequence (SEQ ID NO:5), a p15A origin of replication for E. coli and a pVS1 origin of replication for the replication in P. putida KT2440. The pVS1 origin comes from the Pseudomonas plasmid pVS1 (Itoh Y, et al. Plasmid 1984, 11(3), 206-20). The synthetic DNA fragment was cloned into the vector pACYC {PRhaRS}[rhaRS_Ec]{PRha}[rhlA_Pa rbwB_Srub] using the restriction site PspXI and NEBuilder® HiFi DNA Assembly Master Mix. The assembled product was transformed into NEB® 10-beta Electrocompetent E. coli cells (New England Biolabs, Frankfurt/Main, Germany). Procedure of PCR purification, cloning and transformation were carried out according to manufacturer's manual. The correct insertion of the target genes was checked by restriction analysis and the authenticity of the introduced DNA fragments was verified by DNA sequencing. The resulting plasmid was named pACYC {PRhaRS}[rhaRS_Ec]{PRha}[rhlA_Pa rbwB_Pan997]{ter} (SEQ ID NO:17).

The P. putida strain KT2440 was transformed with the plasmid pACYC {PRhaRS}[rhaRS_Ec]{PRha}[rhlA_Pa rbwB_Pan997]{ter} by means of electroporation (Iwasaki K, et al., Biosci. Biotech. Biochem. 1994. 58(5):851-854)) and plated onto LB-agar plates supplemented with kanamycin (50 μg/mL). Transformants were checked for the presence of the correct plasmid by plasmid preparation and analytical restriction analysis. The resulting strain was named BS-S-647 (P.putida KT2440+pACYC {PRhaRS}[rhaRS_Ec]{PRha}[rhlA_Pa rbwB_Pan997]{ter}).

### Example 5

Construction of an Expression Vector for the P. aeruginosa Gene rhlA and Pantoea sp. SJZ147 Gene rbwB

For the heterologous expression of the genes rhlA_Pa as enzyme E₁ and rbwB_PanSJ as enzyme E₂ from Pantoea sp. SJZ147 the plasmid pACYC {PRhaRS}[rhaRS_Ec]{PRha}[rhlA_Pa rbwB_PanSJ]{ter} was constructed. The synthetic operon consisting of rhlA_Pa (SEQ ID NO:18) which encodes a 3-(3-hydroxyalkanoyloxy)alkanoic acid (HAA) synthase (RhlA, SEQ ID NO:15) and a glucosyltransferase (RbwB, SEQ ID NO:8), respectively, was cloned under the control of the rhamnose inducible promoter Pᵣₕₐ into the vector pACYC {PRhaRS}[rhaRS_Ec]{PRha}[rhlA_Pa rbwB_Srub] (WO2019154984), which is based on pAYCY184 (New England Biolabs, Frankfurt/Main, Germany). Downstream of the synthetic operon a terminator sequence is located. The gene rbwB_PanSJ was synthetically generated by Eurofins Genomics as DNA fragment. The plasmid pACYC {PRhaRS}[rhaRS_Ec]{PRha}[rhlA_Pa rbwB_Srub] carries the P_{Rha} promoter cassette (SEQ ID NO:4) and the terminator sequence (SEQ ID NO:5), a p15A origin of replication for E. coli and a pVS1 origin of replication for the replication in P. putida KT2440. The pVS1 origin comes from the Pseudomonas plasmid pVS1 (Itoh Y, et al. Plasmid 1984, 11(3), 206-20). The synthetic DNA fragment was cloned into the vector pACYC {PRhaRS}[rhaRS_Ec]{PRha}[rhlA_Pa rbwB_Srub] using the restriction site PspXI and NEBuilder® HiFi DNA Assembly Master Mix. The assembled product was transformed into NEB® 10-beta Electrocompetent E. coli cells (New England Biolabs, Frankfurt/Main, Germany). Procedure of PCR purification, cloning and transformation were carried out according to manufacturer's manual. The correct insertion of the target genes was checked by restriction analysis and the authenticity of the introduced DNA fragments was verified by DNA sequencing. The resulting plasmid was named pACYC {PRhaRS}[rhaRS_Ec]{PRha}[rhlA_Pa rbwB_PanSJ]{ter} (SEQ ID NO:19).

The P. putida strain KT2440 was transformed with the plasmid pACYC {PRhaRS}[rhaRS_Ec]{PRha}[rhlA_Pa rbwB_PanSJ]{ter} by means of electroporation (Iwasaki K, et al., Biosci. Biotech. Biochem. 1994. 58(5):851-854)) and plated onto LB-agar plates supplemented with kanamycin (50 μg/mL). Transformants were checked for the presence of the correct plasmid by plasmid preparation and analytical restriction analysis. The resulting strain was named BS-S-648 (P.putida KT2440+pACYC {PRhaRS}[rhaRS_Ec]{PRha}[rhlA_Pa rbwB_PanSJ]{ter}).

### Example 6

Construction of an Expression Vector for the P. aeruginosa Gene rhlA and Pantoea stewartii Strain NS381 Gene rbwB

For the heterologous expression of the genes rhlA_Pa as enzyme E₁ and rbwB_Pst381 as enzyme E₂ from Pantoea stewartii strain NS381 the plasmid pACYC {PRhaRS}[rhaRS_Ec]{PRha}[rhlA_Pa rbwB_Pst381]{ter} was constructed. The synthetic operon consisting of rhlA_Pa (SEQ ID NO:20) which encodes a 3-(3-hydroxyalkanoyloxy)alkanoic acid (HAA) synthase (RhlA, SEQ ID NO:15) and a glucosyltransferase (RbwB, SEQ ID NO:12), respectively, was cloned under the control of the rhamnose inducible promoter Pᵣₕₐ into the vector pACYC {PRhaRS}[rhaRS_Ec]{PRha}[rhlA_Pa rbwB_Srub](WO2019154984), which is based on pAYCY184 (New England Biolabs, Frankfurt/Main, Germany). Downstream of the synthetic operon a terminator sequence is located. The gene rbwB_Pst381 was synthetically generated by Eurofins Genomics as DNA fragment. The plasmid pACYC {PRhaRS}[rhaRS_Ec]{PRha}[rhlA_Pa rbwB_Srub] carries the P_{Rha} promoter cassette (SEQ ID NO:4) and the terminator sequence (SEQ ID NO:5), a p15A origin of replication for E. coli and a pVS1 origin of replication for the replication in P. putida KT2440. The pVS1 origin comes from the Pseudomonas plasmid pVS1 (Itoh Y, et al. Plasmid 1984, 11(3), 206-20). The synthetic DNA fragment was cloned into the vector pACYC {PRhaRS}[rhaRS_Ec]{PRha}[rhlA_Pa rbwB_Srub] using the restriction site PspXI and NEBuilder® HiFi DNA Assembly Master Mix. The assembled product was transformed into NEB® 10-beta Electrocompetent E. coli cells (New England Biolabs, Frankfurt/Main, Germany). Procedure of PCR purification, cloning and transformation were carried out according to manufacturer's manual. The correct insertion of the target genes was checked by restriction analysis and the authenticity of the introduced DNA fragments was verified by DNA sequencing. The resulting plasmid was named pACYC {PRhaRS}[rhaRS_Ec]{PRha}[rhlA_Pa rbwB_Pst381]{ter} (SEQ ID NO:21).

The P. putida strain KT2440 was transformed with the plasmid pACYC {PRhaRS}[rhaRS_Ec]{PRha}[rhlA_Pa rbwB_Pst381]{ter} by means of electroporation (Iwasaki K, et al., Biosci. Biotech. Biochem. 1994. 58(5):851-854)) and plated onto LB-agar plates supplemented with kanamycin (50 μg/mL). Transformants were checked for the presence of the correct plasmid by plasmid preparation and analytical restriction analysis. The resulting strain was named BS-S-649 (P.putida KT2440+pACYC {PRhaRS}[rhaRS_Ec]{PRha}[rhlA_Pa rbwB_Pst381]{ter}).

### Example 7

Production of Rubiwettin with P. putida Derivatives

For the production of rubiwettin we used the BioLector I system (Beckman Coulter Life Sciences, Baesweiler, Germany). The following strains were analyzed:
BS-S-644: P. putida KT2440+pACYC {PRhaRS}[rhaRS_Ec]{PRha}[rbwA_Pan997 rbwB_Pan997]{ter}
BS-S-645: P. putida KT2440+pACYC {PRhaRS}[rhaRS_Ec]{PRha}[rbwA_PanSJ rbwB_PanSJ]{ter}
BS-S-646: P. putida KT2440+pACYC {PRhaRS}[rhaRS_Ec]{PRha}[rbwA_Pst381 rbwB_Pst381]{ter}
BS-S-647: P. putida KT2440+pACYC {PRhaRS}[rhaRS_Ec]{PRha}[rhlA_Pa rbwB_Pan997]{ter}
BS-S-648: P. putida KT2440+pACYC {PRhaRS}[rhaRS_Ec]{PRha}[rhlA_Pa rbwB_PanSJ]{ter}
BS-S-649: P. putida KT2440+pACYC {PRhaRS}[rhaRS_Ec]{PRha}[rhlA_Pa rbwB_Pst381]{ter}

The precultures were inoculated from a glycerol stock in a FlowerPlate without optodes (48 well MTP, flower, Beckman Coulter Life Sciences, Baesweiler, Germany, Art.-No.: MTP-48-B) with 1 ml seed media (autoclaved: 4.4 g/L Na2HPO4*2 H2O, 1.5 g/L KH2PO4, 1 g/L NH4Cl, 10 g/L yeast extract, sterilized separately: 20 g/L glucose, 0.2 g/L MgSO4*7 H2O, 0.006 g/L FeCl3, 0.015 g/L CaCl2, 1 ml/L trace elements solution SL6 (sterile-filtered: 0.3 g/L H3BO3, 0.2 g/L CoCl2×6 H2O, 0.1 g/L ZnSO4×7 H2O, 0.03 g/L MnCl2×4H2O, 0.01 g/L CuCI2×2 H2O, 0.03 g/L Na2MoO4×2 H2O, 0.02 g/L NiCl2×6 H2O) supplemented with kanamycin (50 μg/mL). The precultures were incubated for ~19 h at 1000 rpm and 32° C. and a relative humidity of 85%.

The main cultures were inoculated with the preculture to reach a start OD₆₀₀₀ of 0.5 in a FlowerPlate with pH and dissolved oxygen optodes (48 well MTP, flower, Beckman Coulter Life Sciences, Baesweiler, Germany, Art.-No.: M2P-MTP-48-BOH1) in M12-FIT media (2.2 g/L (NH4)2SO4, 0.02 g/L NaCl, 0.4 g/L MgSO4×7H2O, 0.05 g/L CaCl2×2H2O, 3 g/L KH2PO4, 8.51 g/L Na2HPO4*2H2O, 5 g/L glucose, 25 g/L maltodextrin (C*Dry MD 01955), 10 mL/L trace elements solution M12 (sterile-filtered: 0.2 g/L ZnSO4×7 H2O, 0.1 g/L MnCl2×4H2O, 1.5 g/L Na3-Citrat×2H2O, 0.1 g/L CuSO4×5 H2O, 0.002 g/L NiCl2×6 H2O, 0.003 g/L Na2MoO4×2 H2O, 0.03 g/L H3BO3, 1 g/L FeSO4×7 H2O)) supplemented with kanamycin (50 μg/mL). The main cultures were incubated for ~50 h at 800 rpm and 32° C. and a relative humidity of 85%.

The feed starts by adding 1% (v/v) of sterile-filtered amyloglucosidase solution (100000 U/L) from Aspergillus niger and was triggered via the pO₂ peak which indicates the end of the batch phase. The expression of the target genes was induced with 0.2% (w/v) at the same time. After cultivation we could detect a concentration (mg/L) of rubiwettins with different chain length (see Table 1).

TABLE 1
Rubiwettins concentrations obtained with different P. putida strains. Concentrations
of the individual rubiwettin species with the indicated chain lengths and degree
of saturation of the 3-hydroxyfatty acid portions are indicated.

| | mg/L | mg/L | mg/L | mg/L | mg/L | mg/L | mg/L | mg/L | mg/L | mg/L | mg/L |
|---|---|---|---|---|---|---|---|---|---|---|---|
| | strain | | | | | | | | | | |
| | 1GL- | 1GL- | 1RL- | 1GL- | 1GL- | 1GL- | 1GL- | 1GL- | 1GL- | 1GL- | 1GL- |
| | C8- | C8- | C10- | C10- | C10- | C10- | C12- | C12- | C10- | C10- | C10- |
| | C8 | C10 | C10:1 | C10 | C12:1 | C12 | C12:1 | C12 | C14 | C16:1 | C16 |
| P. putida + | 0 | 0 | 0 | 0 | 0 | 0 | 0 | 0 | 0 | 0 | 0 |
| pACYCATh-5 | | | | | | | | | | | |
| P. putida + | 0 | 0 | 0 | 0 | 0 | 0 | 0 | 0 | 0 | 0 | 0 |
| pACYCATh-5 | | | | | | | | | | | |
| P. putida + | 0 | 0 | 0 | 0 | 0 | 0 | 0 | 0 | 0 | 0 | 0 |
| pACYCATh-5 | | | | | | | | | | | |
| BS-S-644 | 0 | 25 | 0 | 413 | 39 | 66 | 0 | 0 | 0 | 0 | 0 |
| BS-S-644 | 0 | 24 | 0 | 383 | 31 | 59 | 0 | 0 | 0 | 0 | 0 |
| BS-S-644 | 0 | 23 | 0 | 398 | 34 | 66 | 0 | 0 | 0 | 0 | 0 |
| BS-S-645 | 0 | 0 | 0 | 0 | 0 | 0 | 0 | 0 | 0 | 0 | 0 |
| BS-S-645 | 0 | 0 | 0 | 0 | 0 | 0 | 0 | 0 | 0 | 0 | 0 |
| BS-S-645 | 0 | 0 | 0 | 0 | 0 | 0 | 0 | 0 | 0 | 0 | 0 |
| BS-S-646 | 31 | 334 | 19 | 2724 | 218 | 365 | 0 | 0 | 0 | 0 | 0 |
| BS-S-646 | 34 | 317 | 21 | 2697 | 204 | 352 | 0 | 0 | 0 | 0 | 0 |
| BS-S-646 | 27 | 288 | 0 | 2638 | 194 | 350 | 0 | 0 | 0 | 0 | 0 |
| BS-S-647 | 0 | 403 | 20 | 2742 | 82 | 126 | 0 | 0 | 0 | 0 | 0 |
| BS-S-647 | 25 | 346 | 24 | 1915 | 51 | 86 | 0 | 0 | 0 | 0 | 0 |
| BS-S-647 | 28 | 358 | 28 | 2025 | 65 | 82 | 0 | 0 | 0 | 0 | 0 |
| BS-S-648 | 108 | 973 | 42 | 2979 | 175 | 201 | 0 | 0 | 0 | 0 | 0 |
| BS-S-648 | 105 | 978 | 43 | 2989 | 161 | 179 | 0 | 0 | 0 | 0 | 0 |
| BS-S-648 | 107 | 1025 | 42 | 3165 | 172 | 199 | 0 | 0 | 0 | 0 | 0 |
| BS-S-649 | 118 | 1095 | 48 | 3855 | 167 | 205 | 0 | 0 | 0 | 0 | 0 |
| BS-S-649 | 120 | 1104 | 57 | 3991 | 202 | 224 | 0 | 0 | 0 | 0 | 0 |
| BS-S-649 | 127 | 1154 | 57 | 4117 | 197 | 235 | 0 | 0 | 0 | 0 | 0 |

TABLE 2
Proportions of the individual rubiwettin species produced with different P. putida strains.
Proportions of the individual rubiwettin species with the indicated chain lengths and degree
of saturation of the 3-hydroxyfatty acid portions are given in percent of total rubiwettins.

| | % | % | % | % | % | % | % | % | % | % | % |
|---|---|---|---|---|---|---|---|---|---|---|---|
| | strain | | | | | | | | | | |
| | 1GL- | 1GL- | 1RL- | 1GL- | 1GL- | 1GL- | 1GL- | 1GL- | 1GL- | 1GL- | 1GL- |
| | C8- | C8- | C10- | C10- | C10- | C10- | C12- | C12- | C10- | C10- | C10- |
| | C8 | C10 | C10:1 | C10 | C12:1 | C12 | C12:1 | C12 | C14 | C16:1 | C16 |
| P. putida + | 0.0 | 0.0 | 0.0 | 0.0 | 0.0 | 0.0 | 0.0 | 0.0 | 0.0 | 0.0 | 0.0 |
| pACYCATh-5 | | | | | | | | | | | |
| P. putida + | 0.0 | 0.0 | 0.0 | 0.0 | 0.0 | 0.0 | 0.0 | 0.0 | 0.0 | 0.0 | 0.0 |
| pACYCATh-5 | | | | | | | | | | | |
| P. putida + | 0.0 | 0.0 | 0.0 | 0.0 | 0.0 | 0.0 | 0.0 | 0.0 | 0.0 | 0.0 | 0.0 |
| pACYCATh-5 | | | | | | | | | | | |
| BS-S-644 | 0.0 | 4.1 | 0.0 | 68.7 | 6.5 | 11.0 | 0.0 | 0.0 | 0.0 | 0.0 | 0.0 |
| BS-S-644 | 0.0 | 4.3 | 0.0 | 70.1 | 5.7 | 10.8 | 0.0 | 0.0 | 0.0 | 0.0 | 0.0 |
| BS-S-644 | 0.0 | 4.0 | 0.0 | 70.0 | 6.0 | 11.7 | 0.0 | 0.0 | 0.0 | 0.0 | 0.0 |
| BS-S-645 | 0.0 | 0.0 | 0.0 | 0.0 | 0.0 | 0.0 | 0.0 | 0.0 | 0.0 | 0.0 | 0.0 |
| BS-S-645 | 0.0 | 0.0 | 0.0 | 0.0 | 0.0 | 0.0 | 0.0 | 0.0 | 0.0 | 0.0 | 0.0 |
| BS-S-645 | 0.0 | 0.0 | 0.0 | 0.0 | 0.0 | 0.0 | 0.0 | 0.0 | 0.0 | 0.0 | 0.0 |
| BS-S-646 | 0.8 | 8.9 | 0.5 | 72.8 | 5.8 | 9.8 | 0.0 | 0.0 | 0.0 | 0.0 | 0.0 |
| BS-S-646 | 0.9 | 8.6 | 0.6 | 73.3 | 5.5 | 9.6 | 0.0 | 0.0 | 0.0 | 0.0 | 0.0 |
| BS-S-646 | 0.7 | 8.1 | 0.0 | 74.1 | 5.5 | 9.8 | 0.0 | 0.0 | 0.0 | 0.0 | 0.0 |
| BS-S-647 | 0.0 | 11.6 | 0.6 | 79.0 | 2.4 | 3.6 | 0.0 | 0.0 | 0.0 | 0.0 | 0.0 |
| BS-S-647 | 1.0 | 13.6 | 0.9 | 75.2 | 2.0 | 3.4 | 0.0 | 0.0 | 0.0 | 0.0 | 0.0 |
| BS-S-647 | 1.0 | 13.3 | 1.0 | 75.4 | 2.4 | 3.1 | 0.0 | 0.0 | 0.0 | 0.0 | 0.0 |
| BS-S-648 | 2.4 | 21.5 | 0.9 | 66.0 | 3.9 | 4.4 | 0.0 | 0.0 | 0.0 | 0.0 | 0.0 |
| BS-S-648 | 2.3 | 21.8 | 1.0 | 66.5 | 3.6 | 4.0 | 0.0 | 0.0 | 0.0 | 0.0 | 0.0 |
| BS-S-648 | 2.3 | 21.6 | 0.9 | 66.6 | 3.6 | 4.2 | 0.0 | 0.0 | 0.0 | 0.0 | 0.0 |
| BS-S-649 | 2.1 | 19.8 | 0.9 | 69.6 | 3.0 | 3.7 | 0.0 | 0.0 | 0.0 | 0.0 | 0.0 |
| BS-S-649 | 2.1 | 19.2 | 1.0 | 69.4 | 3.5 | 3.9 | 0.0 | 0.0 | 0.0 | 0.0 | 0.0 |
| BS-S-649 | 2.1 | 19.5 | 1.0 | 69.5 | 3.3 | 4.0 | 0.0 | 0.0 | 0.0 | 0.0 | 0.0 |

The results show that the use of SEQ-IDs 1, 7, and 11 from Pantoea spec. as E1 instead of SEQ-ID a from Pseudomonas aeruginosa leads to a shift of rubiwettin species from C10:0-C12:1 and C10:0-012:0 towards 08:0-C10:0. The rubiwettin species with shorter 3-hydroxyfatty acid chain length will have different application profiles than the rubiwettin species with longer 3-hydroxyfatty acid chain length.

### Example 7

Construction of Expression Vectors for the P. aeruginosa Gene Rh1A and rbwB Gene from Serratia rubidaea For the heterologous expression of the gene rhlA_Pa as enzyme E₁ in combination with gene rbwB_Srub from Serratia rubidaea as enzyme E₂, the genes were brought under the control of the rhamnose inducible promoter Pᵣₕₐ using the pACYC5 vector backbone as described in WO2019154984, example 3.

### Example 8

Production of Rubiwettin with P. putida Derivatives

For the production of rubiwettin we used the BioLector I system (Beckman Coulter Life Sciences, Baesweiler, Germany). The following strains were analyzed:
BS-S-368: P. putida KT2440+pACYC_rhlA_Pa_rbwB_Srub (Example 7)
BS-S-647: P. putida KT2440+pACYC {PRhaRS}[rhaRS_Ec]{PRha}[rhlA_Pa rbwB_Pan997]{ter}

### Example 4

BS-S-648: P. putida KT2440+pACYC {PRhaRS}[rhaRS_Ec]{PRha}[rhlA_Pa rbwB_PanSJ]{ter}

### Example 5

BS-S-649: P. putida KT2440+pACYC {PRhaRS}[rhaRS_Ec]{PRha}[rhlA_Pa rbwB_Pst381]{ter}

### Example 6

The precultures were inoculated from a glycerol stock in a FlowerPlate without optodes (48 well MTP, flower, Beckman Coulter Life Sciences, Baesweiler, Germany, Art.-No.: MTP-48-B) with 1 ml seed media (autoclaved: 4.4 g/L Na2HPO4*2 H2O, 1.5 g/L KH2PO4, 1 g/L NH4Cl, 10 g/L yeast extract, sterilized separately: 20 g/L glucose, 0.2 g/L MgSO4*7 H2O, 0.006 g/L FeCl3, 0.015 g/L CaCl2), 1 ml/L trace elements solution SL6 (sterile-filtered: 0.3 g/L H3BO3, 0.2 g/L CoCl2×6 H2O, 0.1 g/L ZnSO4×7 H2O, 0.03 g/L MnCl2×4H2O, 0.01 g/L CuCl2×2 H2O, 0.03 g/L Na2MoO4×2 H2O, 0.02 g/L NiCl2×6 H2O) supplemented with kanamycin (50 μg/mL). The precultures were incubated for ~19 h at 1000 rpm and 32° C. and a relative humidity of 85%.

The main cultures were inoculated with the preculture to reach a start OD₆₀₀ of 0.5 in a FlowerPlate with pH and dissolved oxygen optodes (48 well MTP, flower, Beckman Coulter Life Sciences, Baesweiler, Germany, Art.-No.: M2P-MTP-48-BOH1) in M12-FIT media (2.2 g/L (NH4)2SO4, 0.02 g/L NaCl, 0.4 g/L MgSO4×7H2O, 0.05 g/L CaCl2)×2H2O, 3 g/L KH2PO4, 8.51 g/L Na2HPO4*2H2O, 5 g/L glucose, 25 g/L maltodextrin (C*Dry MD 01955), 10 mL/L trace elements solution M12 (sterile-filtered: 0.2 g/L ZnSO4×7 H2O, 0.1 g/L MnCl2×4H2O, 1.5 g/L Na3-Citrat×2H2O, 0.1 g/L CuSO4×5 H2O, 0.002 g/L NiCl2×6 H2O, 0.003 g/L Na2MoO4×2 H2O, 0.03 g/L H3BO3, 1 g/L FeSO4×7 H2O)) supplemented with kanamycin (50 μg/mL). The main cultures were incubated for ~50 h at 800 rpm and 32° C. and a relative humidity of 85%.

The feed starts by adding 1% (v/v) of sterile-filtered amyloglucosidase solution (100000 U/L) from Aspergillus niger and was triggered via the pO₂ peak which indicates the end of the batch phase. The expression of the target genes was induced with 0.2% (w/v) at the same time. After cultivation we could detect a concentration (mg/L) of rubiwettins with different chain length (see Table 3).

TABLE 3
Rubiwettin concentrations obtained with different P. putida strains. Concentrations
of the individual rubiwettin species with the indicated chain lengths and degree
of saturation of the 3-hydroxyfatty acid portions are indicated.

| | mg/L | mg/L | mg/L | mg/L | mg/L | mg/L | mg/L | mg/L | mg/L | mg/L | mg/L |
|---|---|---|---|---|---|---|---|---|---|---|---|
| | strain | | | | | | | | | | |
| | 1GL- | 1GL- | 1RL- | 1GL- | 1GL- | 1GL- | 1GL- | 1GL- | 1GL- | 1GL- | 1GL- |
| | C8- | C8- | C10- | C10- | C10- | C10- | C12- | C12- | C10- | C10- | C10- |
| | C8 | C10 | C10:1 | C10 | C12:1 | C12 | C12:1 | C12 | C14 | C16:1 | C16 |
| BS-S-368 | 10 | 125 | 12 | 1073 | 27 | 41 | 0 | 0 | 0 | 0 | 0 |
| BS-S-368 | 9 | 111 | 13 | 1175 | 25 | 45 | 0 | 0 | 0 | 0 | 0 |
| BS-S-368 | 9 | 131 | 12 | 1333 | 23 | 60 | 0 | 0 | 0 | 0 | 0 |
| BS-S-647 | 0 | 403 | 20 | 2742 | 82 | 126 | 0 | 0 | 0 | 0 | 0 |
| BS-S-647 | 25 | 346 | 24 | 1915 | 51 | 86 | 0 | 0 | 0 | 0 | 0 |
| BS-S-647 | 28 | 358 | 28 | 2025 | 65 | 82 | 0 | 0 | 0 | 0 | 0 |
| BS-S-648 | 108 | 973 | 42 | 2979 | 175 | 201 | 0 | 0 | 0 | 0 | 0 |
| BS-S-648 | 105 | 978 | 43 | 2989 | 161 | 179 | 0 | 0 | 0 | 0 | 0 |
| BS-S-648 | 107 | 1025 | 42 | 3165 | 172 | 199 | 0 | 0 | 0 | 0 | 0 |
| BS-S-649 | 118 | 1095 | 48 | 3855 | 167 | 205 | 0 | 0 | 0 | 0 | 0 |
| BS-S-649 | 120 | 1104 | 57 | 3991 | 202 | 224 | 0 | 0 | 0 | 0 | 0 |
| BS-S-649 | 127 | 1154 | 57 | 4117 | 197 | 235 | 0 | 0 | 0 | 0 | 0 |

The results show that the use of SEQ ID NO: 2, SEQ ID NO: 8, or SEQ ID NO: 12 from this application originating from Pantoea spec. as enzyme E₂ instead of SEQ ID NO: 4 from WO2019154984 originating from Serratia rubidaea led to production of higher amounts of rubiwettins.

### Example 9

### HPLC-Based Quantification of Rubiwettins

Quantification of lipids R1 and RG1 was carried out by means of HPLC. Using a displacement pipette (Combitip), 400 μl of n-propanol was introduced into a 2 ml reaction vessel and the reaction vessel was immediately closed for minimization of evaporation. The addition of 400 μl fermentation broth followed. After shaking for 1 min in a Retsch mill at a frequency of 30 Hz, the resulting crude extract mixture was centrifuged for 5 min at 13,000 rpm, sterile filtered with an 0.2 μm PVDF filter and 800 μl of the clear supernatant was transferred into an HPLC vial. Further dilutions of cell broth were carried out in 55% (v/v) propanol. Samples were stored at −20° C. before measurement.

For the detection and quantification of lipids an evaporation light scattering detector (Sedex LT-ELSD Model 85LT) was used. The measurement was carried out by means of Agilent Technologies 1200 Series (Santa Clara, Calif.) and a Zorbax SB-C8 Rapid Resolution column (4,6×150 mm, 3.5 μm, Agilent). The injection volume was 5.0 μl and the run time was 20 min. Mobile phase A: aqueous 0.1% TFA (trifluoracetic acid, solution); mobile phase B: methanol. The column temperature was 40° C. The ELSD (detector temperature 60° C.) and the DAD (diode array, 210 nm) were used as detectors.

### Gradient:

| t [min] | | Flow [1 ml/min] |
|---|---|---|
| 0.00 | 70% | 1.00 |
| 15.00 | 100% | 1.00 |
| 15.01 | 70% | 1.00 |
| 20.00 | 70% | 1.00 |

The gradient used starts with 70% B in A to 100% B within 15 minutes at a flow rate of 1 mL/min followed by 5 minutes of re-equilibration with 70% B in A (see Table 2). Reference materials were used whose identity and purity were checked by HPLC-MS/MS and NMR.

1. A microbial cell for producing at least one lipid with general formula II from at least one carbon substrate, 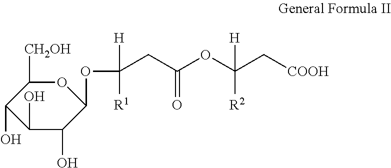
wherein R¹ and R² independently of one another comprises identical or different organic radicals each with 5 to 13 carbon atoms,
wherein the cell is a non-pathogenic cell that is genetically modified to increase the heterologous expression relative to the wild-type cell of:
Enzyme E₁ a 3-(3-hydroxyalkanoyloxy)alkanoic acid (HAA) synthase comprising SEQ ID NO: 1, SEQ ID NO: 7, SEQ ID NO:11, SEQ ID NO: 15 or a variant thereof, wherein the variant comprises 60% sequence identity to SEQ ID NO: 1, 7, 11 or 15; and
Enzyme E₂ a glycosyltransferase (EC 2.4) comprising SEQ ID NO: 2, SEQ ID NO: 8, or SEQ ID NO: 12, or a variant thereof, wherein the variant comprises 60% sequence identity to SEQ ID NO: 2, 8 or 12.

2. The cell according to claim 1, wherein the R in the lipid with general formula II is a saturated alkyl radical.

3. The cell according to claim 2, wherein the alkyl radical is selected from the group consisting of pentyl, heptyl, nonyl, undecyl, and tridecyl.

4. The cell according to claim 1, wherein the cell produces a further lipid with general formula I from the carbon substrate, 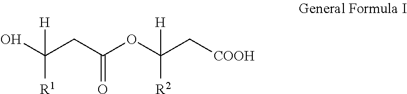
wherein R¹ and R² independently of one another comprises identical or different organic radicals each with 5 to 13 carbon atoms.

5. The cell according to claim 4, wherein the R in the lipid with general formula I is a saturated alkyl radical.

6. The cell according to claim 1, wherein the carbon source is selected from the group consisting of glucose, dextrose, sucrose, xylose, mannose, galactose, arabinose, monosaccharides, polysaccharides, cellulose, hemicelluloses, vegetal oils, animal fats, fatty acids, fatty acid esters, carbonaceous gases, alkanes, glycerol, acetate, ethanol and methanol.

7. The cell according to claim 1, wherein the cell is selected from the group consisting of Acinetobacter sp., Bacillus sp., Brevibacterium sp., Burkholderia sp., Chlorella sp., Clostridium sp., Corynebacterium sp., Cyanobakterien, Escherichia sp., Pseudomonas sp., Klebsiella sp., Salmonella sp., Rhizobium sp., Saccharomyces sp., Pichia sp., and Nostoc sp.

8. The cell according to claim 1, wherein the cell is selected from the group consisting of Bacillus subtilis, Burkholderia thailandensis, Corynebacterium glutamicum, E. coli, Klebsiella oxytoca, Pseudomonas fluorescens, Pseudomonas putida, Pseudomonas stutzeri, Rhizobium meliloti, Saccharomyces cerevisiae and Pichia pastoris.

9. A method of producing at least one lipid with general formula II and/or general formula I: 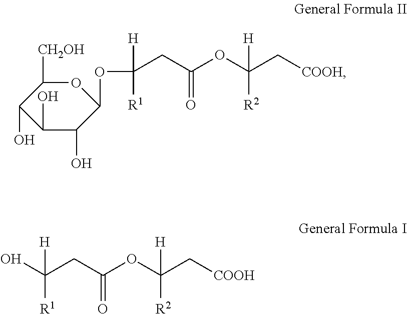
wherein R¹ and R² independently of one another comprises identical or different organic radicals each with 5 to 13 carbon atoms, and
wherein the method comprises a step of contacting at least one cell according to claim 1 with at least one carbon source.

10. The method according to claim 9, wherein the carbon source is selected from the group consisting of glucose, dextrose, sucrose, xylose, mannose, galactose, arabinose, monosaccharides, polysaccharides, cellulose, hemicelluloses, vegetal oils, animal fats, fatty acids, fatty acid esters, carbonaceous gases, alkanes, glycerol, acetate, ethanol and methanol.

11. Use of the cell according to claim 1 for producing at least one lipid with general formula I and/or II: 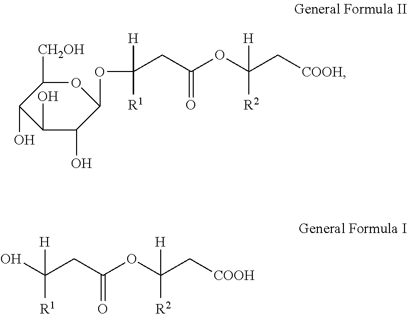
wherein R¹ and R² independently of one another comprises identical or different organic radicals each with 5 to 13 carbon atoms.


SEQUENCE LISTING (21 sequences)

SEQ ID NO: 1 - protein, 278 aa, Pantoea ananatis
MSHAYSVININKLNVFVELVKCSVPTRNRTILINGALATSSSFRNWKRFLADKSDVITFDLPFSGKSKPYNEQDCNIVTLDDEVAIIREIISIYKPNIIASASWGGASTLKLLNNGEDGLEHAIIASYSFEFNDRMRTYVKQANLYSKEKKFVDLAHLMNEEVGAYLPNKMKFCNFRHLTTLDEIEYRQGRFHLEQIANIRNHDYKSIIDNSACQFHFINGDLDIHTPQKNIEEICGESDKTCFYQIRDAGHFLDLEGKVSRERTAAAFNTIFNQIGV

SEQ ID NO: 2 - protein, 390 aa, Pantoea ananatis
MKVIIGALGSAGDVFPCIEIGSLLKEKNHDVYLLANDYFRSEAVSRGLSFVAVGDKETYVNAVQDRSLWEKKHSLKRISHYMAEQQEAMFNAMSALVDDDFIIIHSLWCFAAKAVSDKYQIKKYAISLTNSNLKLQPGRVINALEKGLRVNLNWKLALFQRALVSPALQDTLDKLRRDNHLPACRNIYADWVSGGQRSIVLYEPWFYQKKARNGFYAGFMLNKSRASTDDEQITAFIDSRTVVFFTSWALAEKKSVSQILQDLKAEGLKCVLVTPTATALSVNDDVMTVPQLNIALVETCLFAIHHGGIGTTAQLLKNGVPQLIYPRAFDQFENARALERLACGLKGKGIKEIRKMAALSRHKKHNCDYYAAQFDKAGNKGHELENFLTR

SEQ ID NO: 3 - DNA, 2011 bp, Pantoea ananatis
  source 1..2011: mol_type = genomic DNA
atgagtcacgcatactctgttataaacataaacaaacttaacgtttttgtggaattagtgaaatgcagcgtgcccacacgtaacaggaccatcctgataaatggcgcgctggcgacctcgtcttcctttcggaactggaagcggtttctggcggataaatcagatgtgattacgttcgatctgcctttttccggcaaatcgaaaccctacaatgagcaggactgtaatatcgttacgctggatgatgaagtggcgatcattcgggaaattatttcaatctataagcctaacattattgcctctgcttcgtggggcggcgcatcgacgctgaagctgttaaataacggcgaagatggcctggaacacgccattattgcgtcttactctttcgaatttaacgatcgaatgcgcacctacgttaaacaggcaaatctgtactcaaaagagaaaaaatttgtcgatcttgcgcatttgatgaacgaagaagtcggcgcctatttgccgaacaaaatgaaattctgcaatttcagacacctcaccacgctggatgaaattgagtaccgccaggggcgcttccaccttgagcaaatcgctaatattcgcaatcatgactacaaaagtattatcgataattcggcatgtcagtttcactttatcaatggcgatctggatattcatacgccgcagaaaaacattgaagagatttgcggcgagtcggacaaaacctgcttttatcagatcagagatgcgggacactttttagatctggaaggaaaagttagccgggagcgtacggctgccgcctttaacaccatttttaaccagattggtgtctgaaatgaaagtgatcattggcgcactcggctcagctggcgatgtctttccctgtattgaaattggatcgcttctgaaagaaaaaaatcacgatgtttatttgctcgctaacgattacttcaggtcagaagccgtttctcgcggactttcctttgttgcggtgggcgataaagaaacgtacgtcaacgcggtacaggaccgttcgctgtgggaaaaaaagcactctctgaaacgtatctcacactatatggcggaacagcaggaagccatgttcaatgccatgagcgcccttgtggatgatgacttcattatcattcattctttatggtgcttcgctgcaaaagcggtaagtgacaaataccagattaagaaatatgcaatcagcctgaccaattcaaatttaaaattgcagcccgggcgcgtgattaacgcgctggaaaaaggcctgagagtgaatctgaactggaaactggccttatttcaacgcgcactggtgtcacccgcacttcaggatacgctggacaaattacgccgcgacaatcacctgcccgcctgtcgaaatatttatgctgactgggtgagcggtggacagcgttcgatcgtgttatatgaaccctggttctatcagaaaaaagcgcgtaacgggttttacgctggttttatgcttaataaaagccgtgcatcaacggatgacgaacagattaccgcctttattgactcccggacggtggtgttttttaccagttgggcactggctgagaaaaagtccgttagccagatattgcaggacttaaaagctgaggggttaaaatgcgtacttgttacgccaacagcaaccgccttgtcggtgaacgatgacgtgatgaccgtacctcaactgaatattgcccttgtcgaaacgtgcttatttgccattcatcatggcggtattggcaccaccgcgcagttactcaagaatggcgttccccaactgatctatccccgcgcgttcgatcagtttgaaaacgcccgtgcacttgagcggctggcctgtgggctgaaaggaaaagggataaaggaaatccggaaaatggcggcgctttcacgccataagaaacataattgcgattactacgcggcgcaatttgataaagcgggcaataaaggtcacgaactggaaaactttctgacccgttaa

SEQ ID NO: 4 - DNA, 2025 bp, synthetic construct
  source 1..2025: mol_type = other DNA
atctttctgcgaattgagatgacgccactggctgggcgtcatcccggtttcccgggtaaacaccaccgaaaaatagttactatcttcaaagccacattcggtcgaaatatcactgattaacaggcggctatgctggagaagatattgcgcatgacacactctgacctgtcgcagatattgattgatggtcattccagtctgctggcgaaattgctgacgcaaaacgcgctcactgcacgatgcctcatcacaaaatttatccagcgcaaagggacttttcaggctagccgccagccgggtaatcagcttatccagcaacgtttcgctggatgttggcggcaacgaatcactggtgtaacgatggcgattcagcaacatcaccaactgcccgaacagcaactcagccatttcgttagcaaacggcacatgctgactactttcatgctcaagctgaccgataacctgccgcgcctgcgccatccccatgctacctaagcgccagtgtggttgccctgcgctggcgttaaatcccggaatcgccccctgccagtcaagattcagcttcagacgctccgggcaataaataatattctgcaaaaccagatcgttaacggaagcgtaggagtgtttatcgtcagcatgaatgtaaaagagatcgccacgggtaatgcgataagggcgatcgttgagtacatgcaggccattaccgcgccagacaatcaccagctcacaaaaatcatgtgtatgttcagcaaagacatcttgcggataacggtcagccacagcgactgcctgctggtcgctggcaaaaaaatcatctttgagaagttttaactgatgcgccaccgtggctacctcggccagagaacgaagttgattattcgcaatatggcgtacaaatacgttgagaagattcgcgttattgcagaaagccatcccgtccctggcgaatatcacgcggtgaccagttaaactctcggcgaaaaagcgtcgaaaagtggttactgtcgctgaatccacagcgataggcgatgtcagtaacgctggcctcgctgtggcgtagcagatgtcgggctttcatcagtcgcaggcggttcaggtatcgctgaggcgtcagtcccgtttgctgcttaagctgccgatgtagcgtacgcagtgaaagagaaaattgatccgccacggcatcccaattcacctcatcggcaaaatggtcctccagccaggccagaagcaagttgagacgtgatgcgctgttttccaggttctcctgcaaactgcttttacgcagcaagagcagtaattgcataaacaagatctcgcgactggcggtcgagggtaaatcattttccccttcctgctgttccatctgtgcaaccagctgtcgcacctgctgcaatacgctgtggttaacgcgccagtgagacggatactgcccatccagctcttgtggcagcaactgattcagcccggcgagaaactgaaatcgatccggcgagcgatacagcacattggtcagacacagattatcggtatgttcatacagatgccgatcatgatcgcgtacgaaacagaccgtgccaccggtgatggtatagggctgcccattaaacacatgaatacccgtgccatgttcgacaatcacaatttcatgaaaatcatgatgatgttcaggaaaatccgcctgcgggagccggggttctatcgccacggacgcgttaccagacggaaaaaaatccacactatgtaatacggtcatactggcctcctgatgtcgtcaacacggcgaaatagtaatcacgaggtcaggttcttaccttaaattttcgacggaaaaccacgtaaaaaacgtcgatttttcaagatacagcgtgaattttcaggaaatgcggtgagcatcacatcaccacaattcagcaaattgtgaacatcatcacgttcatctttccctggttgccaatggcccattttcctgtcagtaacgagaaggtcgcgaattcaggcgctttttagactggtcgtaatgaa

SEQ ID NO: 5 - DNA, 107 bp, synthetic construct
  source 1..107: mol_type = other DNA
caaataaaacgaaaggctcagtcgaaagactgggcctttcgttttatctgttgtttgtcggtgaacgctctcctgagtaggacaaatccgccgggagcggatttgaa

SEQ ID NO: 6 - DNA, 11091 bp, synthetic construct
  source 1..11091: mol_type = other DNA
tcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggatttaaatttaatctttctgcgaattgagatgacgccactggctgggcgtcatcccggtttcccgggtaaacaccaccgaaaaatagttactatcttcaaagccacattcggtcgaaatatcactgattaacaggcggctatgctggagaagatattgcgcatgacacactctgacctgtcgcagatattgattgatggtcattccagtctgctggcgaaattgctgacgcaaaacgcgctcactgcacgatgcctcatcacaaaatttatccagcgcaaagggacttttcaggctagccgccagccgggtaatcagcttatccagcaacgtttcgctggatgttggcggcaacgaatcactggtgtaacgatggcgattcagcaacatcaccaactgcccgaacagcaactcagccatttcgttagcaaacggcacatgctgactactttcatgctcaagctgaccgataacctgccgcgcctgcgccatccccatgctacctaagcgccagtgtggttgccctgcgctggcgttaaatcccggaatcgccccctgccagtcaagattcagcttcagacgctccgggcaataaataatattctgcaaaaccagatcgttaacggaagcgtaggagtgtttatcgtcagcatgaatgtaaaagagatcgccacgggtaatgcgataagggcgatcgttgagtacatgcaggccattaccgcgccagacaatcaccagctcacaaaaatcatgtgtatgttcagcaaagacatcttgcggataacggtcagccacagcgactgcctgctggtcgctggcaaaaaaatcatctttgagaagttttaactgatgcgccaccgtggctacctcggccagagaacgaagttgattattcgcaatatggcgtacaaatacgttgagaagattcgcgttattgcagaaagccatcccgtccctggcgaatatcacgcggtgaccagttaaactctcggcgaaaaagcgtcgaaaagtggttactgtcgctgaatccacagcgataggcgatgtcagtaacgctggcctcgctgtggcgtagcagatgtcgggctttcatcagtcgcaggcggttcaggtatcgctgaggcgtcagtcccgtttgctgcttaagctgccgatgtagcgtacgcagtgaaagagaaaattgatccgccacggcatcccaattcacctcatcggcaaaatggtcctccagccaggccagaagcaagttgagacgtgatgcgctgttttccaggttctcctgcaaactgcttttacgcagcaagagcagtaattgcataaacaagatctcgcgactggcggtcgagggtaaatcattttccccttcctgctgttccatctgtgcaaccagctgtcgcacctgctgcaatacgctgtggttaacgcgccagtgagacggatactgcccatccagctcttgtggcagcaactgattcagcccggcgagaaactgaaatcgatccggcgagcgatacagcacattggtcagacacagattatcggtatgttcatacagatgccgatcatgatcgcgtacgaaacagaccgtgccaccggtgatggtatagggctgcccattaaacacatgaatacccgtgccatgttcgacaatcacaatttcatgaaaatcatgatgatgttcaggaaaatccgcctgcgggagccggggttctatcgccacggacgcgttaccagacggaaaaaaatccacactatgtaatacggtcatactggcctcctgatgtcgtcaacacggcgaaatagtaatcacgaggtcaggttcttaccttaaattttcgacggaaaaccacgtaaaaaacgtcgatttttcaagatacagcgtgaattttcaggaaatgcggtgagcatcacatcaccacaattcagcaaattgtgaacatcatcacgttcatctttccctggttgccaatggcccattttcctgtcagtaacgagaaggtcgcgaattcaggcgctttttagactggtcgtaatgaacatttaaatgaattcccttgggactctagagatccgcgggggcccaggaggggggatctggcatttttgggaggtgtgaatgagtcacgcatactctgttataaacataaacaaacttaacgtttttgtggaattagtgaaatgcagcgtgcccacacgtaacaggaccatcctgataaatggcgcgctggcgacctcgtcttcctttcggaactggaagcggtttctggcggataaatcagatgtgattacgttcgatctgcctttttccggcaaatcgaaaccctacaatgagcaggactgtaatatcgttacgctggatgatgaagtggcgatcattcgggaaattatttcaatctataagcctaacattattgcctctgcttcgtggggcggcgcatcgacgctgaagctgttaaataacggcgaagatggcctggaacacgccattattgcgtcttactctttcgaatttaacgatcgaatgcgcacctacgttaaacaggcaaatctgtactcaaaagagaaaaaatttgtcgatcttgcgcatttgatgaacgaagaagtcggcgcctatttgccgaacaaaatgaaattctgcaatttcagacacctcaccacgctggatgaaattgagtaccgccaggggcgcttccaccttgagcaaatcgctaatattcgcaatcatgactacaaaagtattatcgataattcggcatgtcagtttcactttatcaatggcgatctggatattcatacgccgcagaaaaacattgaagagatttgcggcgagtcggacaaaacctgcttttatcagatcagagatgcgggacactttttagatctggaaggaaaagttagccgggagcgtacggctgccgcctttaacaccatttttaaccagattggtgtctgaaatgaaagtgatcattggcgcactcggctcagctggcgatgtctttccctgtattgaaattggatcgcttctgaaagaaaaaaatcacgatgtttatttgctcgctaacgattacttcaggtcagaagccgtttctcgcggactttcctttgttgcggtgggcgataaagaaacgtacgtcaacgcggtacaggaccgttcgctgtgggaaaaaaagcactctctgaaacgtatctcacactatatggcggaacagcaggaagccatgttcaatgccatgagcgcccttgtggatgatgacttcattatcattcattctttatggtgcttcgctgcaaaagcggtaagtgacaaataccagattaagaaatatgcaatcagcctgaccaattcaaatttaaaattgcagcccgggcgcgtgattaacgcgctggaaaaaggcctgagagtgaatctgaactggaaactggccttatttcaacgcgcactggtgtcacccgcacttcaggatacgctggacaaattacgccgcgacaatcacctgcccgcctgtcgaaatatttatgctgactgggtgagcggtggacagcgttcgatcgtgttatatgaaccctggttctatcagaaaaaagcgcgtaacgggttttacgctggttttatgcttaataaaagccgtgcatcaacggatgacgaacagattaccgcctttattgactcccggacggtggtgttttttaccagttgggcactggctgagaaaaagtccgttagccagatattgcaggacttaaaagctgaggggttaaaatgcgtacttgttacgccaacagcaaccgccttgtcggtgaacgatgacgtgatgaccgtacctcaactgaatattgcccttgtcgaaacgtgcttatttgccattcatcatggcggtattggcaccaccgcgcagttactcaagaatggcgttccccaactgatctatccccgcgcgttcgatcagtttgaaaacgcccgtgcacttgagcggctggcctgtgggctgaaaggaaaagggataaaggaaatccggaaaatggcggcgctttcacgccataagaaacataattgcgattactacgcggcgcaatttgataaagcgggcaataaaggtcacgaactggaaaactttctgacccgttaagctcgagcacgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgatgataagctgtcaaacatgagaattcttgaagacgaaagggcctcgtgtgtacaaacgttcgtcaaaagggcgacacaaaattcctgcaggggccggcccagcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcaactagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttccggatgagcattca

SEQ ID NO: 7 - protein, 269 aa, Pantoea sp.
MSHECSVLNINELNVYVELTKCSVPTRNRTILINGALATSSSFRNWKRFLADKSDVITFDLPFSGKSKPFNEQDCNIVTLDDEVAIIREIISIYKPNIIASASWGGASTLKLLHNGEEGLEHAIIASYSFEFNDRMRTYVKQANLYSKEKKFVDLAHLMNEEVGAYLPNKMKFCNFRHLTTLDEIEYRQGRFHLEQISNIRNHDYKRIIDNSACQFHFINGDLDIHTPQKNIEEICGESDKTRFYQIRECGTLFRPGRKGQPGANRRRL

SEQ ID NO: 8 - protein, 390 aa, Pantoea sp.
MKVIIGALGSAGDVFPCIEVGSLLKEKHHDVYLLANDYFRSEAVSRGLSFVAVGDKETYVNAVQDRSLWEKKHSLKRISYYMAEQQEAMFDAMSALVDDDCIIIHSLWCFAAKAVSDKYQIKKYAISLTNSNLKLQPGRLINALEKSFKVNLNWKLALFQRALVSPTLQDTIDKLRRDNNLPACRNIYADWVSSEQHTIVLYESWFYQKKARDGFYAGFLLNKSRASSDDDRITAFIDSRTVVFFTSWALAEKKSVAPILQDLKDEGLKCVLVTPTAKALSVKDDVMTVPQLNIALAETCLFAIHHGGIGTTAQLLKNGVPQLIYPRAFDQFENARALERLACGLKGKGIGEIRKMAALSRQKKHNCDFYAAQLDQQNNKGQELERFLTR

SEQ ID NO: 9 - DNA, 2012 bp, Pantoea sp.
  source 1..2012: mol_type = genomic DNA
atgagtcacgaatgttccgtcctaaacataaatgaacttaacgtttatgtggagttaacaaaatgcagcgttcctacgcgtaacaggaccatcctgatcaatggtgcgctggcaacgtcttcttcatttcgcaactggaaacggttcctggcagacaaatccgatgtgattaccttcgatttaccgttttctggcaaatcgaagcctttcaacgagcaggactgtaatatcgtcacgctggatgatgaagtggccatcatccgggaaattatttcaatctataagccgaacattatcgcttccgcctcatggggcggcgcatccacgctgaagctgttacataacggcgaagagggcctggaacacgccattattgcgtcttactctttcgaatttaacgatcgtatgcgcacctacgttaagcaggcaaacctgtactcgaaagagaaaaagtttgtcgatcttgcccatttgatgaatgaggaagtgggtgcgtatttgccgaataaaatgaaattctgcaacttcaggcacctgaccacgctggatgagattgagtaccggcaggggcgtttccacctggagcaaatctctaacattcgcaatcatgactacaagcgcattatcgataattcggcatgtcagtttcacttcatcaatggcgatctggatattcatactccacagaaaaacattgaagagatatgcggggaatcagataaaacacgcttttatcaaatcagagaatgcgggacactttttagacctggaaggaaaggtcagccgggagcgaaccgccgccgcctttaataccatttttagtcaaattggtgtctgaaatgaaagtgattattggcgctctgggctcggcaggtgatgtttttccctgtattgaagtgggttcgctcctgaaagagaaacatcacgatgtttacctgctcgctaacgactatttcaggtcagaagcggtgtctcgcggactttcctttgttgccgtcggcgataaagaaacctacgtgaacgcagtacaggatcgatccttatgggaaaagaagcattctttaaaacggatttcgtactatatggcggaacagcaggaagccatgttcgatgccatgagcgcccttgtcgacgatgactgcatcatcattcattctttatggtgctttgcggcgaaagccgtaagcgataaatatcagataaagaaatacgcaatcagcctgaccaattcaaatttaaaattgcagcccggacgactcatcaacgcgctggaaaaaagctttaaggtaaatttgaactggaagctggccttatttcaacgagcgctggtttcacccacgcttcaggacacaatagacaaattacgccgcgacaataacctgcccgcttgtcgtaatatttatgccgattgggtaagcagcgaacagcatacaatcgtgttatatgaatcctggttctatcaaaaaaaggcgcgcgacggattttatgcgggctttctgcttaacaaaagtcgcgcatcctcggatgacgatcgcattactgcttttatcgactcgcgcacggtggtcttttttaccagttgggcgctggcggagaaaaaatcggttgccccgatattgcaggatttaaaggatgagggactgaagtgcgttcttgttacgccaacggcaaaagcgttatcggtaaaagatgacgttatgaccgtgcctcagttgaatattgcgctggcagaaacgtgcttatttgccattcatcatggcggcattgggacgaccgcgcagttactcaaaaatggcgtgccccaactcatctatcctcgcgcgttcgatcagtttgaaaatgcccgtgccctggaacggttggcctgtggactgaaaggaaaagggatcggggagatcagaaaaatggcggcgctttcacgtcaaaagaaacacaattgcgacttttatgccgcgcaacttgatcaacaaaacaataaaggccaggaactggaacgctttctgacccgttaa

SEQ ID NO: 10 - DNA, 11092 bp, synthetic construct
  source 1..11092: mol_type = other DNA
tcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggatttaaatttaatctttctgcgaattgagatgacgccactggctgggcgtcatcccggtttcccgggtaaacaccaccgaaaaatagttactatcttcaaagccacattcggtcgaaatatcactgattaacaggcggctatgctggagaagatattgcgcatgacacactctgacctgtcgcagatattgattgatggtcattccagtctgctggcgaaattgctgacgcaaaacgcgctcactgcacgatgcctcatcacaaaatttatccagcgcaaagggacttttcaggctagccgccagccgggtaatcagcttatccagcaacgtttcgctggatgttggcggcaacgaatcactggtgtaacgatggcgattcagcaacatcaccaactgcccgaacagcaactcagccatttcgttagcaaacggcacatgctgactactttcatgctcaagctgaccgataacctgccgcgcctgcgccatccccatgctacctaagcgccagtgtggttgccctgcgctggcgttaaatcccggaatcgccccctgccagtcaagattcagcttcagacgctccgggcaataaataatattctgcaaaaccagatcgttaacggaagcgtaggagtgtttatcgtcagcatgaatgtaaaagagatcgccacgggtaatgcgataagggcgatcgttgagtacatgcaggccattaccgcgccagacaatcaccagctcacaaaaatcatgtgtatgttcagcaaagacatcttgcggataacggtcagccacagcgactgcctgctggtcgctggcaaaaaaatcatctttgagaagttttaactgatgcgccaccgtggctacctcggccagagaacgaagttgattattcgcaatatggcgtacaaatacgttgagaagattcgcgttattgcagaaagccatcccgtccctggcgaatatcacgcggtgaccagttaaactctcggcgaaaaagcgtcgaaaagtggttactgtcgctgaatccacagcgataggcgatgtcagtaacgctggcctcgctgtggcgtagcagatgtcgggctttcatcagtcgcaggcggttcaggtatcgctgaggcgtcagtcccgtttgctgcttaagctgccgatgtagcgtacgcagtgaaagagaaaattgatccgccacggcatcccaattcacctcatcggcaaaatggtcctccagccaggccagaagcaagttgagacgtgatgcgctgttttccaggttctcctgcaaactgcttttacgcagcaagagcagtaattgcataaacaagatctcgcgactggcggtcgagggtaaatcattttccccttcctgctgttccatctgtgcaaccagctgtcgcacctgctgcaatacgctgtggttaacgcgccagtgagacggatactgcccatccagctcttgtggcagcaactgattcagcccggcgagaaactgaaatcgatccggcgagcgatacagcacattggtcagacacagattatcggtatgttcatacagatgccgatcatgatcgcgtacgaaacagaccgtgccaccggtgatggtatagggctgcccattaaacacatgaatacccgtgccatgttcgacaatcacaatttcatgaaaatcatgatgatgttcaggaaaatccgcctgcgggagccggggttctatcgccacggacgcgttaccagacggaaaaaaatccacactatgtaatacggtcatactggcctcctgatgtcgtcaacacggcgaaatagtaatcacgaggtcaggttcttaccttaaattttcgacggaaaaccacgtaaaaaacgtcgatttttcaagatacagcgtgaattttcaggaaatgcggtgagcatcacatcaccacaattcagcaaattgtgaacatcatcacgttcatctttccctggttgccaatggcccattttcctgtcagtaacgagaaggtcgcgaattcaggcgctttttagactggtcgtaatgaacatttaaatgaattcccttgggactctagagatccgcgggggcccaggaggggggatctggcatttttgggaggtgtgaatgagtcacgaatgttccgtcctaaacataaatgaacttaacgtttatgtggagttaacaaaatgcagcgttcctacgcgtaacaggaccatcctgatcaatggtgcgctggcaacgtcttcttcatttcgcaactggaaacggttcctggcagacaaatccgatgtgattaccttcgatttaccgttttctggcaaatcgaagcctttcaacgagcaggactgtaatatcgtcacgctggatgatgaagtggccatcatccgggaaattatttcaatctataagccgaacattatcgcttccgcctcatggggcggcgcatccacgctgaagctgttacataacggcgaagagggcctggaacacgccattattgcgtcttactctttcgaatttaacgatcgtatgcgcacctacgttaagcaggcaaacctgtactcgaaagagaaaaagtttgtcgatcttgcccatttgatgaatgaggaagtgggtgcgtatttgccgaataaaatgaaattctgcaacttcaggcacctgaccacgctggatgagattgagtaccggcaggggcgtttccacctggagcaaatctctaacattcgcaatcatgactacaagcgcattatcgataattcggcatgtcagtttcacttcatcaatggcgatctggatattcatactccacagaaaaacattgaagagatatgcggggaatcagataaaacacgcttttatcaaatcagagaatgcgggacactttttagacctggaaggaaaggtcagccgggagcgaaccgccgccgcctttaataccatttttagtcaaattggtgtctgaaatgaaagtgattattggcgctctgggctcggcaggtgatgtttttccctgtattgaagtgggttcgctcctgaaagagaaacatcacgatgtttacctgctcgctaacgactatttcaggtcagaagcggtgtctcgcggactttcctttgttgccgtcggcgataaagaaacctacgtgaacgcagtacaggatcgatccttatgggaaaagaagcattctttaaaacggatttcgtactatatggcggaacagcaggaagccatgttcgatgccatgagcgcccttgtcgacgatgactgcatcatcattcattctttatggtgctttgcggcgaaagccgtaagcgataaatatcagataaagaaatacgcaatcagcctgaccaattcaaatttaaaattgcagcccggacgactcatcaacgcgctggaaaaaagctttaaggtaaatttgaactggaagctggccttatttcaacgagcgctggtttcacccacgcttcaggacacaatagacaaattacgccgcgacaataacctgcccgcttgtcgtaatatttatgccgattgggtaagcagcgaacagcatacaatcgtgttatatgaatcctggttctatcaaaaaaaggcgcgcgacggattttatgcgggctttctgcttaacaaaagtcgcgcatcctcggatgacgatcgcattactgcttttatcgactcgcgcacggtggtcttttttaccagttgggcgctggcggagaaaaaatcggttgccccgatattgcaggatttaaaggatgagggactgaagtgcgttcttgttacgccaacggcaaaagcgttatcggtaaaagatgacgttatgaccgtgcctcagttgaatattgcgctggcagaaacgtgcttatttgccattcatcatggcggcattgggacgaccgcgcagttactcaaaaatggcgtgccccaactcatctatcctcgcgcgttcgatcagtttgaaaatgcccgtgccctggaacggttggcctgtggactgaaaggaaaagggatcggggagatcagaaaaatggcggcgctttcacgtcaaaagaaacacaattgcgacttttatgccgcgcaacttgatcaacaaaacaataaaggccaggaactggaacgctttctgacccgttaagctcgagcacgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgatgataagctgtcaaacatgagaattcttgaagacgaaagggcctcgtgtgtacaaacgttcgtcaaaagggcgacacaaaattcctgcaggggccggcccagcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcaactagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttccggatgagcattca

SEQ ID NO: 11 - protein, 278 aa, Pantoea stewartii
MSHGYSVVNINELNIYVELTKCSVPTRNRTILINGALATSSSFRNWKRFLADKSDVITFDLPFSGKSKPFNEQGCHIVTLDDEVTIIRELISIYKPNIIASASWGGASTLKLLNHGEEGLEHAIIASYSFEFNDRMRTYVKQANLFSKEKNFVELAHLMNDEVGAYLPNKMKFCNFRHLTSLDEIEYRQGRFHLEQIATLRNHDYKNIIDNSACQFHFINGDLDIHTPQKNIEEICGESDKTRFYQIRDAGHFLDLEGKRSRERTAAAFNTIFSQIGV

SEQ ID NO: 12 - protein, 390 aa, Pantoea stewartii
MKVIIGALGSAGDVFPCIEIGSLLKEKNHDVYLLANDYFRSEAVDRGLSFVAVGDKETYVKAVQDRALWDKKHALKRISHYMAEQQEAMFDAMSALVEDDAIIIHSLWCFAAKAVSEKYRLKKYAISLTNSNLKLQPGRVIHALEKSLRVSLNWKLALFQRALVSPALQRTIDTLRRDHNLPASRNIYADWVNNEPQSIVLYEPWFYQKKAREGFYAGFLLNKSRASLDDDRITDFIDSRTVVFFTSWALAEQSSVTRVLQDLKTEGLKCVLVTPTAAALAVNDDVMTLPHLNIALTETCLFAIHHGGIGTTAQLLKNGVPQLIYPCAFDQFENARALERLACGLKGKGIREIRKMAALARQKKHNCDFYAVQLANQTHRGLELERFLTC

SEQ ID NO: 13 - DNA, 2011 bp, Pantoea stewartii
  source 1..2011: mol_type = genomic DNA
atgagtcacggttattctgtagtaaacataaatgaacttaacatttatgtggagttaacaaaatgcagtgtcccgacccgtaacaggaccatcctgattaacggtgcactggcaacctcgtcttctttccgcaactggaaacgcttcctggcggataaatcagatgtcattacgtttgatcttccgttctcaggcaaatcaaaacccttcaacgaacagggctgccatatcgtcacgcttgatgatgaagtcacgattattcgcgagctgatttcaatttataagcccaatattattgcctcagcctcctggggcggcgcatcgactctcaagctcttgaatcacggtgaagagggcctggaacatgccatcatcgcgtcctattcctttgaatttaatgaccgtatgcggacttacgtgaagcaagccaatctgttttcaaaagagaagaactttgtcgagcttgcgcatttgatgaatgacgaagtgggtgcatatctgccgaataaaatgaagttctgcaacttcaggcatctgacgtcgctggatgagattgaataccgtcaggggcgtttccatcttgagcagattgccacgctgcgcaaccacgactacaaaaacattattgataactcggcctgccagtttcactttatcaacggcgacctggatattcatacgccgcaaaaaaacatcgaagagatatgcggcgagtcggataaaacgcgcttttatcaaatcagggatgcgggacattttctggatctggaaggcaagcgcagccgggaaaggaccgcagccgccttcaacactatttttagtcagattggtgtctgagatgaaagtgattattggcgcactgggctcagcaggcgatgtctttccctgtattgagattggctcgctgttaaaagaaaagaatcatgatgtttacctgctcgctaacgattatttcagatctgaggcagtagatcgcggcctgtcctttgtggcggtcggcgacaaagagacttatgtgaaagccgtccaggatcgggcgttatgggacaaaaagcacgccctgaaacggatttcacattatatggcggaacagcaggaggcgatgttcgatgccatgagcgctcttgtcgaagatgacgccatcatcattcactctttatggtgttttgcggcgaaagccgtcagcgagaaataccggttaaagaaatatgctatcagcctgaccaattcaaatttaaaattgcagcccgggcgtgttatccatgcgctggaaaaaagcctgcgtgtcagtctgaactggaaactggctttatttcaacgggccctggtttcgcctgcactccagcgtaccatcgacacgctgcgacgcgaccataatctgcccgcctctcgcaatatttacgctgactgggtcaataacgaaccgcagtcgattgtgttatacgaaccctggttttatcaaaagaaagcgcgcgagggattttatgccggttttttactgaacaagagccgggcgtcgctggatgacgatcgtattaccgattttattgactcccgtacggtggtcttttttaccagttgggccctggctgaacaatcatccgtgacacgcgttttacaggacttaaaaacagaaggactgaagtgcgtgcttgttacgccaaccgcagccgcgttagcggtaaacgatgatgtgatgacgctgccgcatctcaatattgccctgactgaaacctgcttatttgccattcatcatggcggtattggcaccaccgcgcagttgctcaagaacggggtgcctcaactgatttatccgtgcgcatttgatcagtttgaaaatgcccgcgcccttgaacgactggcctgcggcctcaagggaaaagggatcagggaaatcagaaaaatggcggcacttgcacgtcagaagaaacacaactgcgacttttatgctgtgcaactggctaatcaaacccacagaggtcttgagctggaacgctttctgacctgttaa

SEQ ID NO: 14 - DNA, 11092 bp, synthetic construct
  source 1..11092: mol_type = other DNA
tcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggatttaaatttaatctttctgcgaattgagatgacgccactggctgggcgtcatcccggtttcccgggtaaacaccaccgaaaaatagttactatcttcaaagccacattcggtcgaaatatcactgattaacaggcggctatgctggagaagatattgcgcatgacacactctgacctgtcgcagatattgattgatggtcattccagtctgctggcgaaattgctgacgcaaaacgcgctcactgcacgatgcctcatcacaaaatttatccagcgcaaagggacttttcaggctagccgccagccgggtaatcagcttatccagcaacgtttcgctggatgttggcggcaacgaatcactggtgtaacgatggcgattcagcaacatcaccaactgcccgaacagcaactcagccatttcgttagcaaacggcacatgctgactactttcatgctcaagctgaccgataacctgccgcgcctgcgccatccccatgctacctaagcgccagtgtggttgccctgcgctggcgttaaatcccggaatcgccccctgccagtcaagattcagcttcagacgctccgggcaataaataatattctgcaaaaccagatcgttaacggaagcgtaggagtgtttatcgtcagcatgaatgtaaaagagatcgccacgggtaatgcgataagggcgatcgttgagtacatgcaggccattaccgcgccagacaatcaccagctcacaaaaatcatgtgtatgttcagcaaagacatcttgcggataacggtcagccacagcgactgcctgctggtcgctggcaaaaaaatcatctttgagaagttttaactgatgcgccaccgtggctacctcggccagagaacgaagttgattattcgcaatatggcgtacaaatacgttgagaagattcgcgttattgcagaaagccatcccgtccctggcgaatatcacgcggtgaccagttaaactctcggcgaaaaagcgtcgaaaagtggttactgtcgctgaatccacagcgataggcgatgtcagtaacgctggcctcgctgtggcgtagcagatgtcgggctttcatcagtcgcaggcggttcaggtatcgctgaggcgtcagtcccgtttgctgcttaagctgccgatgtagcgtacgcagtgaaagagaaaattgatccgccacggcatcccaattcacctcatcggcaaaatggtcctccagccaggccagaagcaagttgagacgtgatgcgctgttttccaggttctcctgcaaactgcttttacgcagcaagagcagtaattgcataaacaagatctcgcgactggcggtcgagggtaaatcattttccccttcctgctgttccatctgtgcaaccagctgtcgcacctgctgcaatacgctgtggttaacgcgccagtgagacggatactgcccatccagctcttgtggcagcaactgattcagcccggcgagaaactgaaatcgatccggcgagcgatacagcacattggtcagacacagattatcggtatgttcatacagatgccgatcatgatcgcgtacgaaacagaccgtgccaccggtgatggtatagggctgcccattaaacacatgaatacccgtgccatgttcgacaatcacaatttcatgaaaatcatgatgatgttcaggaaaatccgcctgcgggagccggggttctatcgccacggacgcgttaccagacggaaaaaaatccacactatgtaatacggtcatactggcctcctgatgtcgtcaacacggcgaaatagtaatcacgaggtcaggttcttaccttaaattttcgacggaaaaccacgtaaaaaacgtcgatttttcaagatacagcgtgaattttcaggaaatgcggtgagcatcacatcaccacaattcagcaaattgtgaacatcatcacgttcatctttccctggttgccaatggcccattttcctgtcagtaacgagaaggtcgcgaattcaggcgctttttagactggtcgtaatgaacatttaaatgaattcccttgggactctagagatccgcgggggcccaggaggggggatctggcatttttgggaggtgtgaaatgagtcacggttattctgtagtaaacataaatgaacttaacatttatgtggagttaacaaaatgcagtgtcccgacccgtaacaggaccatcctgattaacggtgcactggcaacctcgtcttctttccgcaactggaaacgcttcctggcggataaatcagatgtcattacgtttgatcttccgttctcaggcaaatcaaaacccttcaacgaacagggctgccatatcgtcacgcttgatgatgaagtcacgattattcgcgagctgatttcaatttataagcccaatattattgcctcagcctcctggggcggcgcatcgactctcaagctcttgaatcacggtgaagagggcctggaacatgccatcatcgcgtcctattcctttgaatttaatgaccgtatgcggacttacgtgaagcaagccaatctgttttcaaaagagaagaactttgtcgagcttgcgcatttgatgaatgacgaagtgggtgcatatctgccgaataaaatgaagttctgcaacttcaggcatctgacgtcgctggatgagattgaataccgtcaggggcgtttccatcttgagcagattgccacgctgcgcaaccacgactacaaaaacattattgataactcggcctgccagtttcactttatcaacggcgacctggatattcatacgccgcaaaaaaacatcgaagagatatgcggcgagtcggataaaacgcgcttttatcaaatcagggatgcgggacattttctggatctggaaggcaagcgcagccgggaaaggaccgcagccgccttcaacactatttttagtcagattggtgtctgagatgaaagtgattattggcgcactgggctcagcaggcgatgtctttccctgtattgagattggctcgctgttaaaagaaaagaatcatgatgtttacctgctcgctaacgattatttcagatctgaggcagtagatcgcggcctgtcctttgtggcggtcggcgacaaagagacttatgtgaaagccgtccaggatcgggcgttatgggacaaaaagcacgccctgaaacggatttcacattatatggcggaacagcaggaggcgatgttcgatgccatgagcgctcttgtcgaagatgacgccatcatcattcactctttatggtgttttgcggcgaaagccgtcagcgagaaataccggttaaagaaatatgctatcagcctgaccaattcaaatttaaaattgcagcccgggcgtgttatccatgcgctggaaaaaagcctgcgtgtcagtctgaactggaaactggctttatttcaacgggccctggtttcgcctgcactccagcgtaccatcgacacgctgcgacgcgaccataatctgcccgcctctcgcaatatttacgctgactgggtcaataacgaaccgcagtcgattgtgttatacgaaccctggttttatcaaaagaaagcgcgcgagggattttatgccggttttttactgaacaagagccgggcgtcgctggatgacgatcgtattaccgattttattgactcccgtacggtggtcttttttaccagttgggccctggctgaacaatcatccgtgacacgcgttttacaggacttaaaaacagaaggactgaagtgcgtgcttgttacgccaaccgcagccgcgttagcggtaaacgatgatgtgatgacgctgccgcatctcaatattgccctgactgaaacctgcttatttgccattcatcatggcggtattggcaccaccgcgcagttgctcaagaacggggtgcctcaactgatttatccgtgcgcatttgatcagtttgaaaatgcccgcgcccttgaacgactggcctgcggcctcaagggaaaagggatcagggaaatcagaaaaatggcggcacttgcacgtcagaagaaacacaactgcgacttttatgctgtgcaactggctaatcaaacccacagaggtcttgagctggaacgctttctgacctgttaagctcgagcacgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgatgataagctgtcaaacatgagaattcttgaagacgaaagggcctcgtgtgtacaaacgttcgtcaaaagggcgacacaaaattcctgcaggggccggcccagcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcaactagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttccggatgagcattca

SEQ ID NO: 15 - protein, 295 aa, Pseudomonas aeruginosa
MRRESLLVSVCKGLRVHVERVGQDPGRSTVMLVNGAMATTASFARTCKCLAEHFNVVLFDLPFAGQSRQHNPQRGLITKDDEVEILLALIERFEVNHLVSASWGGISTLLALSRNPRGIRSSVVMAFAPGLNQAMLDYVGRAQALIELDDKSAIGHLLNETVGKYLPPRLKASNHQHMASLATGEYEQARFHIDQVLALNDRGYLACLERIQSHVHFINGSWDEYTTAEDARQFRDYLPHCSFSRVEGTGHFLDLESKLAAVRVHRALLEHLLKQPEPQRAERAAGFHEMAIGYA

SEQ ID NO: 16 - DNA, 2105 bp, synthetic construct
  source 1..2105: mol_type = other DNA
atgcggcgcgaaagtctgttggtatcggtttgcaagggcctgcgggtacatgtcgagcgcgttgggcaggatcccgggcgcagcacggtgatgctggtcaacggcgcgatggcgaccaccgcctcgttcgcccggacctgcaagtgcctggccgaacatttcaacgtggtgctgttcgacctgcccttcgccgggcagtcgcgtcagcacaacccgcagcgggggttgatcaccaaggacgacgaggtggaaatcctcctggcgctgatcgagcgcttcgaggtcaatcacctggtctccgcgtcctggggcggtatctccacgctgctggcgctgtcgcgcaatccgcgcggcatccgcagctcggtggtgatggcattcgcccctggactgaaccaggcgatgctcgactacgtcgggcgggcgcaggcgctgatcgagctggacgacaagtcggcgatcggccatctgctcaacgagaccgtcggcaaatacctgccgccgcgcctgaaagccagcaaccatcagcacatggcttcgctggccaccggcgaatacgagcaggcgcgctttcacatcgaccaggtgctggcgctcaacgatcggggctacctggcttgcctggagcggatccagagccacgtgcatttcatcaacggcagctgggacgaatacaccaccgccgaggacgcccgccagttccgcgactacctgccgcactgcagtttctcgcgggtggagggcaccgggcatttcctcgacctggagtccaagctggccgcggtacgcgtgcaccgcgccctgctcgagcacctgctgaagcaaccggagccgcagcgggcggaacgcgcggcgggattccacgagatggccatcggctacgcctgaggataacaaactaaaatattgattcgacaagacaggatggtgatatgaaagtgatcattggcgcactcggctcagctggcgatgtctttccctgtattgaaattggatcgcttctgaaagaaaaaaatcacgatgtttatttgctcgctaacgattacttcaggtcagaagccgtttctcgcggactttcctttgttgcggtgggcgataaagaaacgtacgtcaacgcggtacaggaccgttcgctgtgggaaaaaaagcactctctgaaacgtatctcacactatatggcggaacagcaggaagccatgttcaatgccatgagcgcccttgtggatgatgacttcattatcattcattctttatggtgcttcgctgcaaaagcggtaagtgacaaataccagattaagaaatatgcaatcagcctgaccaattcaaatttaaaattgcagcccgggcgcgtgattaacgcgctggaaaaaggcctgagagtgaatctgaactggaaactggccttatttcaacgcgcactggtgtcacccgcacttcaggatacgctggacaaattacgccgcgacaatcacctgcccgcctgtcgaaatatttatgctgactgggtgagcggtggacagcgttcgatcgtgttatatgaaccctggttctatcagaaaaaagcgcgtaacgggttttacgctggttttatgcttaataaaagccgtgcatcaacggatgacgaacagattaccgcctttattgactcccggacggtggtgttttttaccagttgggcactggctgagaaaaagtccgttagccagatattgcaggacttaaaagctgaggggttaaaatgcgtacttgttacgccaacagcaaccgccttgtcggtgaacgatgacgtgatgaccgtacctcaactgaatattgcccttgtcgaaacgtgcttatttgccattcatcatggcggtattggcaccaccgcgcagttactcaagaatggcgttccccaactgatctatccccgcgcgttcgatcagtttgaaaacgcccgtgcacttgagcggctggcctgtgggctgaaaggaaaagggataaaggaaatccggaaaatggcggcgctttcacgccataagaaacataattgcgattactacgcggcgcaatttgataaagcgggcaataaaggtcacgaactggaaaactttctgacccgttaa

SEQ ID NO: 17 - DNA, 11186 bp, synthetic construct
  source 1..11186: mol_type = other DNA
tcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggatttaaatttaatctttctgcgaattgagatgacgccactggctgggcgtcatcccggtttcccgggtaaacaccaccgaaaaatagttactatcttcaaagccacattcggtcgaaatatcactgattaacaggcggctatgctggagaagatattgcgcatgacacactctgacctgtcgcagatattgattgatggtcattccagtctgctggcgaaattgctgacgcaaaacgcgctcactgcacgatgcctcatcacaaaatttatccagcgcaaagggacttttcaggctagccgccagccgggtaatcagcttatccagcaacgtttcgctggatgttggcggcaacgaatcactggtgtaacgatggcgattcagcaacatcaccaactgcccgaacagcaactcagccatttcgttagcaaacggcacatgctgactactttcatgctcaagctgaccgataacctgccgcgcctgcgccatccccatgctacctaagcgccagtgtggttgccctgcgctggcgttaaatcccggaatcgccccctgccagtcaagattcagcttcagacgctccgggcaataaataatattctgcaaaaccagatcgttaacggaagcgtaggagtgtttatcgtcagcatgaatgtaaaagagatcgccacgggtaatgcgataagggcgatcgttgagtacatgcaggccattaccgcgccagacaatcaccagctcacaaaaatcatgtgtatgttcagcaaagacatcttgcggataacggtcagccacagcgactgcctgctggtcgctggcaaaaaaatcatctttgagaagttttaactgatgcgccaccgtggctacctcggccagagaacgaagttgattattcgcaatatggcgtacaaatacgttgagaagattcgcgttattgcagaaagccatcccgtccctggcgaatatcacgcggtgaccagttaaactctcggcgaaaaagcgtcgaaaagtggttactgtcgctgaatccacagcgataggcgatgtcagtaacgctggcctcgctgtggcgtagcagatgtcgggctttcatcagtcgcaggcggttcaggtatcgctgaggcgtcagtcccgtttgctgcttaagctgccgatgtagcgtacgcagtgaaagagaaaattgatccgccacggcatcccaattcacctcatcggcaaaatggtcctccagccaggccagaagcaagttgagacgtgatgcgctgttttccaggttctcctgcaaactgcttttacgcagcaagagcagtaattgcataaacaagatctcgcgactggcggtcgagggtaaatcattttccccttcctgctgttccatctgtgcaaccagctgtcgcacctgctgcaatacgctgtggttaacgcgccagtgagacggatactgcccatccagctcttgtggcagcaactgattcagcccggcgagaaactgaaatcgatccggcgagcgatacagcacattggtcagacacagattatcggtatgttcatacagatgccgatcatgatcgcgtacgaaacagaccgtgccaccggtgatggtatagggctgcccattaaacacatgaatacccgtgccatgttcgacaatcacaatttcatgaaaatcatgatgatgttcaggaaaatccgcctgcgggagccggggttctatcgccacggacgcgttaccagacggaaaaaaatccacactatgtaatacggtcatactggcctcctgatgtcgtcaacacggcgaaatagtaatcacgaggtcaggttcttaccttaaattttcgacggaaaaccacgtaaaaaacgtcgatttttcaagatacagcgtgaattttcaggaaatgcggtgagcatcacatcaccacaattcagcaaattgtgaacatcatcacgttcatctttccctggttgccaatggcccattttcctgtcagtaacgagaaggtcgcgaattcaggcgctttttagactggtcgtaatgaacatttaaatgaattcccttgggactctagagatccgcgggggcccaggaggggggatctggcatttttgggaggtgtgaaatgcggcgcgaaagtctgttggtatcggtttgcaagggcctgcgggtacatgtcgagcgcgttgggcaggatcccgggcgcagcacggtgatgctggtcaacggcgcgatggcgaccaccgcctcgttcgcccggacctgcaagtgcctggccgaacatttcaacgtggtgctgttcgacctgcccttcgccgggcagtcgcgtcagcacaacccgcagcgggggttgatcaccaaggacgacgaggtggaaatcctcctggcgctgatcgagcgcttcgaggtcaatcacctggtctccgcgtcctggggcggtatctccacgctgctggcgctgtcgcgcaatccgcgcggcatccgcagctcggtggtgatggcattcgcccctggactgaaccaggcgatgctcgactacgtcgggcgggcgcaggcgctgatcgagctggacgacaagtcggcgatcggccatctgctcaacgagaccgtcggcaaatacctgccgccgcgcctgaaagccagcaaccatcagcacatggcttcgctggccaccggcgaatacgagcaggcgcgctttcacatcgaccaggtgctggcgctcaacgatcggggctacctggcttgcctggagcggatccagagccacgtgcatttcatcaacggcagctgggacgaatacaccaccgccgaggacgcccgccagttccgcgactacctgccgcactgcagtttctcgcgggtggagggcaccgggcatttcctcgacctggagtccaagctggccgcggtacgcgtgcaccgcgccctgctcgagcacctgctgaagcaaccggagccgcagcgggcggaacgcgcggcgggattccacgagatggccatcggctacgcctgaggataacaaactaaaatattgattcgacaagacaggatggtgatatgaaagtgatcattggcgcactcggctcagctggcgatgtctttccctgtattgaaattggatcgcttctgaaagaaaaaaatcacgatgtttatttgctcgctaacgattacttcaggtcagaagccgtttctcgcggactttcctttgttgcggtgggcgataaagaaacgtacgtcaacgcggtacaggaccgttcgctgtgggaaaaaaagcactctctgaaacgtatctcacactatatggcggaacagcaggaagccatgttcaatgccatgagcgcccttgtggatgatgacttcattatcattcattctttatggtgcttcgctgcaaaagcggtaagtgacaaataccagattaagaaatatgcaatcagcctgaccaattcaaatttaaaattgcagcccgggcgcgtgattaacgcgctggaaaaaggcctgagagtgaatctgaactggaaactggccttatttcaacgcgcactggtgtcacccgcacttcaggatacgctggacaaattacgccgcgacaatcacctgcccgcctgtcgaaatatttatgctgactgggtgagcggtggacagcgttcgatcgtgttatatgaaccctggttctatcagaaaaaagcgcgtaacgggttttacgctggttttatgcttaataaaagccgtgcatcaacggatgacgaacagattaccgcctttattgactcccggacggtggtgttttttaccagttgggcactggctgagaaaaagtccgttagccagatattgcaggacttaaaagctgaggggttaaaatgcgtacttgttacgccaacagcaaccgccttgtcggtgaacgatgacgtgatgaccgtacctcaactgaatattgcccttgtcgaaacgtgcttatttgccattcatcatggcggtattggcaccaccgcgcagttactcaagaatggcgttccccaactgatctatccccgcgcgttcgatcagtttgaaaacgcccgtgcacttgagcggctggcctgtgggctgaaaggaaaagggataaaggaaatccggaaaatggcggcgctttcacgccataagaaacataattgcgattactacgcggcgcaatttgataaagcgggcaataaaggtcacgaactggaaaactttctgacccgttaagctcgagcacgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgatgataagctgtcaaacatgagaattcttgaagacgaaagggcctcgtgtgtacaaacgttcgtcaaaagggcgacacaaaattcctgcaggggccggcccagcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcaactagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttccggatgagcattca

SEQ ID NO: 18 - DNA, 2105 bp, synthetic construct
  source 1..2105: mol_type = other DNA
atgcggcgcgaaagtctgttggtatcggtttgcaagggcctgcgggtacatgtcgagcgcgttgggcaggatcccgggcgcagcacggtgatgctggtcaacggcgcgatggcgaccaccgcctcgttcgcccggacctgcaagtgcctggccgaacatttcaacgtggtgctgttcgacctgcccttcgccgggcagtcgcgtcagcacaacccgcagcgggggttgatcaccaaggacgacgaggtggaaatcctcctggcgctgatcgagcgcttcgaggtcaatcacctggtctccgcgtcctggggcggtatctccacgctgctggcgctgtcgcgcaatccgcgcggcatccgcagctcggtggtgatggcattcgcccctggactgaaccaggcgatgctcgactacgtcgggcgggcgcaggcgctgatcgagctggacgacaagtcggcgatcggccatctgctcaacgagaccgtcggcaaatacctgccgccgcgcctgaaagccagcaaccatcagcacatggcttcgctggccaccggcgaatacgagcaggcgcgctttcacatcgaccaggtgctggcgctcaacgatcggggctacctggcttgcctggagcggatccagagccacgtgcatttcatcaacggcagctgggacgaatacaccaccgccgaggacgcccgccagttccgcgactacctgccgcactgcagtttctcgcgggtggagggcaccgggcatttcctcgacctggagtccaagctggccgcggtacgcgtgcaccgcgccctgctcgagcacctgctgaagcaaccggagccgcagcgggcggaacgcgcggcgggattccacgagatggccatcggctacgcctgaggataacaaactaaaatattgattcgacaagacaggatggtgatatgaaagtgattattggcgctctgggctcggcaggtgatgtttttccctgtattgaagtgggttcgctcctgaaagagaaacatcacgatgtttacctgctcgctaacgactatttcaggtcagaagcggtgtctcgcggactttcctttgttgccgtcggcgataaagaaacctacgtgaacgcagtacaggatcgatccttatgggaaaagaagcattctttaaaacggatttcgtactatatggcggaacagcaggaagccatgttcgatgccatgagcgcccttgtcgacgatgactgcatcatcattcattctttatggtgctttgcggcgaaagccgtaagcgataaatatcagataaagaaatacgcaatcagcctgaccaattcaaatttaaaattgcagcccggacgactcatcaacgcgctggaaaaaagctttaaggtaaatttgaactggaagctggccttatttcaacgagcgctggtttcacccacgcttcaggacacaatagacaaattacgccgcgacaataacctgcccgcttgtcgtaatatttatgccgattgggtaagcagcgaacagcatacaatcgtgttatatgaatcctggttctatcaaaaaaaggcgcgcgacggattttatgcgggctttctgcttaacaaaagtcgcgcatcctcggatgacgatcgcattactgcttttatcgactcgcgcacggtggtcttttttaccagttgggcgctggcggagaaaaaatcggttgccccgatattgcaggatttaaaggatgagggactgaagtgcgttcttgttacgccaacggcaaaagcgttatcggtaaaagatgacgttatgaccgtgcctcagttgaatattgcgctggcagaaacgtgcttatttgccattcatcatggcggcattgggacgaccgcgcagttactcaaaaatggcgtgccccaactcatctatcctcgcgcgttcgatcagtttgaaaatgcccgtgccctggaacggttggcctgtggactgaaaggaaaagggatcggggagatcagaaaaatggcggcgctttcacgtcaaaagaaacacaattgcgacttttatgccgcgcaacttgatcaacaaaacaataaaggccaggaactggaacgctttctgacccgttaa

SEQ ID NO: 19 - DNA, 11186 bp, synthetic construct
  source 1..11186: mol_type = other DNA
tcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggatttaaatttaatctttctgcgaattgagatgacgccactggctgggcgtcatcccggtttcccgggtaaacaccaccgaaaaatagttactatcttcaaagccacattcggtcgaaatatcactgattaacaggcggctatgctggagaagatattgcgcatgacacactctgacctgtcgcagatattgattgatggtcattccagtctgctggcgaaattgctgacgcaaaacgcgctcactgcacgatgcctcatcacaaaatttatccagcgcaaagggacttttcaggctagccgccagccgggtaatcagcttatccagcaacgtttcgctggatgttggcggcaacgaatcactggtgtaacgatggcgattcagcaacatcaccaactgcccgaacagcaactcagccatttcgttagcaaacggcacatgctgactactttcatgctcaagctgaccgataacctgccgcgcctgcgccatccccatgctacctaagcgccagtgtggttgccctgcgctggcgttaaatcccggaatcgccccctgccagtcaagattcagcttcagacgctccgggcaataaataatattctgcaaaaccagatcgttaacggaagcgtaggagtgtttatcgtcagcatgaatgtaaaagagatcgccacgggtaatgcgataagggcgatcgttgagtacatgcaggccattaccgcgccagacaatcaccagctcacaaaaatcatgtgtatgttcagcaaagacatcttgcggataacggtcagccacagcgactgcctgctggtcgctggcaaaaaaatcatctttgagaagttttaactgatgcgccaccgtggctacctcggccagagaacgaagttgattattcgcaatatggcgtacaaatacgttgagaagattcgcgttattgcagaaagccatcccgtccctggcgaatatcacgcggtgaccagttaaactctcggcgaaaaagcgtcgaaaagtggttactgtcgctgaatccacagcgataggcgatgtcagtaacgctggcctcgctgtggcgtagcagatgtcgggctttcatcagtcgcaggcggttcaggtatcgctgaggcgtcagtcccgtttgctgcttaagctgccgatgtagcgtacgcagtgaaagagaaaattgatccgccacggcatcccaattcacctcatcggcaaaatggtcctccagccaggccagaagcaagttgagacgtgatgcgctgttttccaggttctcctgcaaactgcttttacgcagcaagagcagtaattgcataaacaagatctcgcgactggcggtcgagggtaaatcattttccccttcctgctgttccatctgtgcaaccagctgtcgcacctgctgcaatacgctgtggttaacgcgccagtgagacggatactgcccatccagctcttgtggcagcaactgattcagcccggcgagaaactgaaatcgatccggcgagcgatacagcacattggtcagacacagattatcggtatgttcatacagatgccgatcatgatcgcgtacgaaacagaccgtgccaccggtgatggtatagggctgcccattaaacacatgaatacccgtgccatgttcgacaatcacaatttcatgaaaatcatgatgatgttcaggaaaatccgcctgcgggagccggggttctatcgccacggacgcgttaccagacggaaaaaaatccacactatgtaatacggtcatactggcctcctgatgtcgtcaacacggcgaaatagtaatcacgaggtcaggttcttaccttaaattttcgacggaaaaccacgtaaaaaacgtcgatttttcaagatacagcgtgaattttcaggaaatgcggtgagcatcacatcaccacaattcagcaaattgtgaacatcatcacgttcatctttccctggttgccaatggcccattttcctgtcagtaacgagaaggtcgcgaattcaggcgctttttagactggtcgtaatgaacatttaaatgaattcccttgggactctagagatccgcgggggcccaggaggggggatctggcatttttgggaggtgtgaaatgcggcgcgaaagtctgttggtatcggtttgcaagggcctgcgggtacatgtcgagcgcgttgggcaggatcccgggcgcagcacggtgatgctggtcaacggcgcgatggcgaccaccgcctcgttcgcccggacctgcaagtgcctggccgaacatttcaacgtggtgctgttcgacctgcccttcgccgggcagtcgcgtcagcacaacccgcagcgggggttgatcaccaaggacgacgaggtggaaatcctcctggcgctgatcgagcgcttcgaggtcaatcacctggtctccgcgtcctggggcggtatctccacgctgctggcgctgtcgcgcaatccgcgcggcatccgcagctcggtggtgatggcattcgcccctggactgaaccaggcgatgctcgactacgtcgggcgggcgcaggcgctgatcgagctggacgacaagtcggcgatcggccatctgctcaacgagaccgtcggcaaatacctgccgccgcgcctgaaagccagcaaccatcagcacatggcttcgctggccaccggcgaatacgagcaggcgcgctttcacatcgaccaggtgctggcgctcaacgatcggggctacctggcttgcctggagcggatccagagccacgtgcatttcatcaacggcagctgggacgaatacaccaccgccgaggacgcccgccagttccgcgactacctgccgcactgcagtttctcgcgggtggagggcaccgggcatttcctcgacctggagtccaagctggccgcggtacgcgtgcaccgcgccctgctcgagcacctgctgaagcaaccggagccgcagcgggcggaacgcgcggcgggattccacgagatggccatcggctacgcctgaggataacaaactaaaatattgattcgacaagacaggatggtgatatgaaagtgattattggcgctctgggctcggcaggtgatgtttttccctgtattgaagtgggttcgctcctgaaagagaaacatcacgatgtttacctgctcgctaacgactatttcaggtcagaagcggtgtctcgcggactttcctttgttgccgtcggcgataaagaaacctacgtgaacgcagtacaggatcgatccttatgggaaaagaagcattctttaaaacggatttcgtactatatggcggaacagcaggaagccatgttcgatgccatgagcgcccttgtcgacgatgactgcatcatcattcattctttatggtgctttgcggcgaaagccgtaagcgataaatatcagataaagaaatacgcaatcagcctgaccaattcaaatttaaaattgcagcccggacgactcatcaacgcgctggaaaaaagctttaaggtaaatttgaactggaagctggccttatttcaacgagcgctggtttcacccacgcttcaggacacaatagacaaattacgccgcgacaataacctgcccgcttgtcgtaatatttatgccgattgggtaagcagcgaacagcatacaatcgtgttatatgaatcctggttctatcaaaaaaaggcgcgcgacggattttatgcgggctttctgcttaacaaaagtcgcgcatcctcggatgacgatcgcattactgcttttatcgactcgcgcacggtggtcttttttaccagttgggcgctggcggagaaaaaatcggttgccccgatattgcaggatttaaaggatgagggactgaagtgcgttcttgttacgccaacggcaaaagcgttatcggtaaaagatgacgttatgaccgtgcctcagttgaatattgcgctggcagaaacgtgcttatttgccattcatcatggcggcattgggacgaccgcgcagttactcaaaaatggcgtgccccaactcatctatcctcgcgcgttcgatcagtttgaaaatgcccgtgccctggaacggttggcctgtggactgaaaggaaaagggatcggggagatcagaaaaatggcggcgctttcacgtcaaaagaaacacaattgcgacttttatgccgcgcaacttgatcaacaaaacaataaaggccaggaactggaacgctttctgacccgttaagctcgagcacgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgatgataagctgtcaaacatgagaattcttgaagacgaaagggcctcgtgtgtacaaacgttcgtcaaaagggcgacacaaaattcctgcaggggccggcccagcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcaactagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttccggatgagcattca

SEQ ID NO: 20 - DNA, 2105 bp, synthetic construct
  source 1..2105: mol_type = other DNA
atgcggcgcgaaagtctgttggtatcggtttgcaagggcctgcgggtacatgtcgagcgcgttgggcaggatcccgggcgcagcacggtgatgctggtcaacggcgcgatggcgaccaccgcctcgttcgcccggacctgcaagtgcctggccgaacatttcaacgtggtgctgttcgacctgcccttcgccgggcagtcgcgtcagcacaacccgcagcgggggttgatcaccaaggacgacgaggtggaaatcctcctggcgctgatcgagcgcttcgaggtcaatcacctggtctccgcgtcctggggcggtatctccacgctgctggcgctgtcgcgcaatccgcgcggcatccgcagctcggtggtgatggcattcgcccctggactgaaccaggcgatgctcgactacgtcgggcgggcgcaggcgctgatcgagctggacgacaagtcggcgatcggccatctgctcaacgagaccgtcggcaaatacctgccgccgcgcctgaaagccagcaaccatcagcacatggcttcgctggccaccggcgaatacgagcaggcgcgctttcacatcgaccaggtgctggcgctcaacgatcggggctacctggcttgcctggagcggatccagagccacgtgcatttcatcaacggcagctgggacgaatacaccaccgccgaggacgcccgccagttccgcgactacctgccgcactgcagtttctcgcgggtggagggcaccgggcatttcctcgacctggagtccaagctggccgcggtacgcgtgcaccgcgccctgctcgagcacctgctgaagcaaccggagccgcagcgggcggaacgcgcggcgggattccacgagatggccatcggctacgcctgaggataacaaactaaaatattgattcgacaagacaggatggtgatatgaaagtgattattggcgcactgggctcagcaggcgatgtctttccctgtattgagattggctcgctgttaaaagaaaagaatcatgatgtttacctgctcgctaacgattatttcagatctgaggcagtagatcgcggcctgtcctttgtggcggtcggcgacaaagagacttatgtgaaagccgtccaggatcgggcgttatgggacaaaaagcacgccctgaaacggatttcacattatatggcggaacagcaggaggcgatgttcgatgccatgagcgctcttgtcgaagatgacgccatcatcattcactctttatggtgttttgcggcgaaagccgtcagcgagaaataccggttaaagaaatatgctatcagcctgaccaattcaaatttaaaattgcagcccgggcgtgttatccatgcgctggaaaaaagcctgcgtgtcagtctgaactggaaactggctttatttcaacgggccctggtttcgcctgcactccagcgtaccatcgacacgctgcgacgcgaccataatctgcccgcctctcgcaatatttacgctgactgggtcaataacgaaccgcagtcgattgtgttatacgaaccctggttttatcaaaagaaagcgcgcgagggattttatgccggttttttactgaacaagagccgggcgtcgctggatgacgatcgtattaccgattttattgactcccgtacggtggtcttttttaccagttgggccctggctgaacaatcatccgtgacacgcgttttacaggacttaaaaacagaaggactgaagtgcgtgcttgttacgccaaccgcagccgcgttagcggtaaacgatgatgtgatgacgctgccgcatctcaatattgccctgactgaaacctgcttatttgccattcatcatggcggtattggcaccaccgcgcagttgctcaagaacggggtgcctcaactgatttatccgtgcgcatttgatcagtttgaaaatgcccgcgcccttgaacgactggcctgcggcctcaagggaaaagggatcagggaaatcagaaaaatggcggcacttgcacgtcagaagaaacacaactgcgacttttatgctgtgcaactggctaatcaaacccacagaggtcttgagctggaacgctttctgacctgttaa

SEQ ID NO: 21 - DNA, 11186 bp, synthetic construct
  source 1..11186: mol_type = other DNA
tcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcattggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggatttaaatttaatctttctgcgaattgagatgacgccactggctgggcgtcatcccggtttcccgggtaaacaccaccgaaaaatagttactatcttcaaagccacattcggtcgaaatatcactgattaacaggcggctatgctggagaagatattgcgcatgacacactctgacctgtcgcagatattgattgatggtcattccagtctgctggcgaaattgctgacgcaaaacgcgctcactgcacgatgcctcatcacaaaatttatccagcgcaaagggacttttcaggctagccgccagccgggtaatcagcttatccagcaacgtttcgctggatgttggcggcaacgaatcactggtgtaacgatggcgattcagcaacatcaccaactgcccgaacagcaactcagccatttcgttagcaaacggcacatgctgactactttcatgctcaagctgaccgataacctgccgcgcctgcgccatccccatgctacctaagcgccagtgtggttgccctgcgctggcgttaaatcccggaatcgccccctgccagtcaagattcagcttcagacgctccgggcaataaataatattctgcaaaaccagatcgttaacggaagcgtaggagtgtttatcgtcagcatgaatgtaaaagagatcgccacgggtaatgcgataagggcgatcgttgagtacatgcaggccattaccgcgccagacaatcaccagctcacaaaaatcatgtgtatgttcagcaaagacatcttgcggataacggtcagccacagcgactgcctgctggtcgctggcaaaaaaatcatctttgagaagttttaactgatgcgccaccgtggctacctcggccagagaacgaagttgattattcgcaatatggcgtacaaatacgttgagaagattcgcgttattgcagaaagccatcccgtccctggcgaatatcacgcggtgaccagttaaactctcggcgaaaaagcgtcgaaaagtggttactgtcgctgaatccacagcgataggcgatgtcagtaacgctggcctcgctgtggcgtagcagatgtcgggctttcatcagtcgcaggcggttcaggtatcgctgaggcgtcagtcccgtttgctgcttaagctgccgatgtagcgtacgcagtgaaagagaaaattgatccgccacggcatcccaattcacctcatcggcaaaatggtcctccagccaggccagaagcaagttgagacgtgatgcgctgttttccaggttctcctgcaaactgcttttacgcagcaagagcagtaattgcataaacaagatctcgcgactggcggtcgagggtaaatcattttccccttcctgctgttccatctgtgcaaccagctgtcgcacctgctgcaatacgctgtggttaacgcgccagtgagacggatactgcccatccagctcttgtggcagcaactgattcagcccggcgagaaactgaaatcgatccggcgagcgatacagcacattggtcagacacagattatcggtatgttcatacagatgccgatcatgatcgcgtacgaaacagaccgtgccaccggtgatggtatagggctgcccattaaacacatgaatacccgtgccatgttcgacaatcacaatttcatgaaaatcatgatgatgttcaggaaaatccgcctgcgggagccggggttctatcgccacggacgcgttaccagacggaaaaaaatccacactatgtaatacggtcatactggcctcctgatgtcgtcaacacggcgaaatagtaatcacgaggtcaggttcttaccttaaattttcgacggaaaaccacgtaaaaaacgtcgatttttcaagatacagcgtgaattttcaggaaatgcggtgagcatcacatcaccacaattcagcaaattgtgaacatcatcacgttcatctttccctggttgccaatggcccattttcctgtcagtaacgagaaggtcgcgaattcaggcgctttttagactggtcgtaatgaacatttaaatgaattcccttgggactctagagatccgcgggggcccaggaggggggatctggcatttttgggaggtgtgaaatgcggcgcgaaagtctgttggtatcggtttgcaagggcctgcgggtacatgtcgagcgcgttgggcaggatcccgggcgcagcacggtgatgctggtcaacggcgcgatggcgaccaccgcctcgttcgcccggacctgcaagtgcctggccgaacatttcaacgtggtgctgttcgacctgcccttcgccgggcagtcgcgtcagcacaacccgcagcgggggttgatcaccaaggacgacgaggtggaaatcctcctggcgctgatcgagcgcttcgaggtcaatcacctggtctccgcgtcctggggcggtatctccacgctgctggcgctgtcgcgcaatccgcgcggcatccgcagctcggtggtgatggcattcgcccctggactgaaccaggcgatgctcgactacgtcgggcgggcgcaggcgctgatcgagctggacgacaagtcggcgatcggccatctgctcaacgagaccgtcggcaaatacctgccgccgcgcctgaaagccagcaaccatcagcacatggcttcgctggccaccggcgaatacgagcaggcgcgctttcacatcgaccaggtgctggcgctcaacgatcggggctacctggcttgcctggagcggatccagagccacgtgcatttcatcaacggcagctgggacgaatacaccaccgccgaggacgcccgccagttccgcgactacctgccgcactgcagtttctcgcgggtggagggcaccgggcatttcctcgacctggagtccaagctggccgcggtacgcgtgcaccgcgccctgctcgagcacctgctgaagcaaccggagccgcagcgggcggaacgcgcggcgggattccacgagatggccatcggctacgcctgaggataacaaactaaaatattgattcgacaagacaggatggtgatatgaaagtgattattggcgcactgggctcagcaggcgatgtctttccctgtattgagattggctcgctgttaaaagaaaagaatcatgatgtttacctgctcgctaacgattatttcagatctgaggcagtagatcgcggcctgtcctttgtggcggtcggcgacaaagagacttatgtgaaagccgtccaggatcgggcgttatgggacaaaaagcacgccctgaaacggatttcacattatatggcggaacagcaggaggcgatgttcgatgccatgagcgctcttgtcgaagatgacgccatcatcattcactctttatggtgttttgcggcgaaagccgtcagcgagaaataccggttaaagaaatatgctatcagcctgaccaattcaaatttaaaattgcagcccgggcgtgttatccatgcgctggaaaaaagcctgcgtgtcagtctgaactggaaactggctttatttcaacgggccctggtttcgcctgcactccagcgtaccatcgacacgctgcgacgcgaccataatctgcccgcctctcgcaatatttacgctgactgggtcaataacgaaccgcagtcgattgtgttatacgaaccctggttttatcaaaagaaagcgcgcgagggattttatgccggttttttactgaacaagagccgggcgtcgctggatgacgatcgtattaccgattttattgactcccgtacggtggtcttttttaccagttgggccctggctgaacaatcatccgtgacacgcgttttacaggacttaaaaacagaaggactgaagtgcgtgcttgttacgccaaccgcagccgcgttagcggtaaacgatgatgtgatgacgctgccgcatctcaatattgccctgactgaaacctgcttatttgccattcatcatggcggtattggcaccaccgcgcagttgctcaagaacggggtgcctcaactgatttatccgtgcgcatttgatcagtttgaaaatgcccgcgcccttgaacgactggcctgcggcctcaagggaaaagggatcagggaaatcagaaaaatggcggcacttgcacgtcagaagaaacacaactgcgacttttatgctgtgcaactggctaatcaaacccacagaggtcttgagctggaacgctttctgacctgttaagctcgagcacgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgatgataagctgtcaaacatgagaattcttgaagacgaaagggcctcgtgtgtacaaacgttcgtcaaaagggcgacacaaaattcctgcaggggccggcccagcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcaactagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttccggatgagcattca